(19) United States
(12) Patent Application Publication
(10) Pub. No.: US 2025/0347654 A1
(43) Pub. Date: Nov. 13, 2025

(54) DETECTION OF AN ANALYTE OF INTEREST BY A CHIP BASED NANOESI DETECTION SYSTEM

(71) Applicant: Roche Diagnostics Operations, Inc., Indianapolis, IN (US)

(72) Inventors: Martin Rempt, Penzberg (DE); Manuel Josef Seitz, Penzberg (DE); Christoph Zuth, Penzberg (DE)

(21) Appl. No.: 19/225,672

(22) Filed: Jun. 2, 2025

Related U.S. Application Data
(63) Continuation of application No. PCT/EP2023/083791, filed on Nov. 30, 2023.

(30) Foreign Application Priority Data
Dec. 2, 2022 (EP) 22211164.3

(51) Int. Cl. G01N 27/623; G01N 1/28; G01N 35/00

(52) U.S. Cl. CPC G01N 27/623; G01N 1/28; G01N 35/00; G01N 2035/00465

(57) ABSTRACT

The present invention relates to a method, a diagnostic system, a kit and the use thereof for efficiently detection of an analyte of interest by a chip based nanoESI detection system.

### CROSS-REFERENCE TO RELATED APPLICATIONS

This application is a continuation of International PCT Application No. PCT/EP2023/083791 filed on Nov. 30, 2023, which claims priority to European Patent Application No. 22211164.3 filed on Dec. 2, 2022, the contents of each application are incorporated herein by reference in their entireties.

### FIELD OF THE INVENTION

The present invention relates to a method, a diagnostic system, a kit and the use thereof for efficiently detection of an analyte of interest by a chip based nanoESI detection system.

### BACKGROUND OF THE INVENTION

Mass spectrometry (MS) is a widely used technique for the qualitative and quantitative analysis of chemical substances ranging from small molecules to macromolecules. In general, it is a very sensitive and specific method, allowing even for the analysis of complex biological, for example (e.g.), environmental or clinical samples. However, for several analytes, especially if analysed from complex biological matrices such as serum, sensitivity of the measurement remains an issue.

Often MS is combined with chromatographic techniques, particularly gas and liquid chromatography such as e.g. HPLC. Here, the analysed molecule (analyte) of interest is separated chromatographically and is individually subjected to mass spectrometric analysis. However, stand-alone mass spectrometry has made substantial progress in selectivity and sensitivity for direct MS detection methods. Unlike the traditional workflows classified into sample preparation, chromatographic separation and mass spectrometric detection, many sample preparation techniques are directly coupled to stand-alone MS exhibiting exceptional performance.

To ensure reliable and sensitive mass spectrometric detection (avoiding matrix effects and interference as well as increasing sensitivity) it is necessary to separate chromatographically the target analytes as well as possible. In general, this can be done by isocratic or gradient systems, for example, reversed phase HPLC columns and gradients from aqueous to organic phases. The columns used for HPLC require flow rates between 0.1 and 1.0 ml/min. Under these optimal flow conditions, very narrow chromatographic peaks with very small peak volumes are produces.

Liquid extraction surface analysis (LESA) mass spectrometry is a direct surface sampling technique. Analytes are extracted from the surface via a liquid micro-junction between a pipette tip and the sample surface. This approach enables sampling of a variety of biological analytes, such as drugs, lipids and proteins, from a range of solid surfaces prior to electrospray ionisation (ESI). Substrates analyzed by LESA include thin tissue sections, bacterial colonies grown on agar, dried blood spots on card and polymeric surfaces. Other direct analysis approaches that have been applied to the analysis of dried blood spots include desorption electrospray ionisation (DESI), direct analysis in real time (DART) and paperspray.

Currently, there are some approaches described in literature that couples bead based solid supports with nano-ESI-MS analysis. Most of them are based on microfluidic chips or devices.

It is known that the importance of nanoESI mass spectrometry has grown during the last years. However, coupling a direct surface sampling technique with nanoESI-MS demands a significantly improved sample preparation technique to reach sensitive, adaptable and fast measurements out of biological matrices. Applying solid support based sample preparation techniques in combination with nanoESI-MS could overcome those hurdles.

Nevertheless, current approaches are not applicable for an efficient detection of an analyte of interest, e.g. for high-throughput measurements, as the described microfluidic devices are difficult to produce in high quantities and do contribute to increased complexity itself.

Combining solid supported sample preparation with nano-ESI mass spectrometric analysis is limited by a complex design and production of solid support containing microfluidic devices, devices that are designed for specific applications with a lack of broad analyte menu, an adaption of the sample preparation that influence the ionization, the system requirement of fluidic or liquid chromatographic parts, low throughput of most examples from literature and/or sample carry-over that influences the measurement.

There is, however, still a need of increasing the efficiency of MS analysis methods, in particular a method which allows for a efficiency detection of analytes from complex biological matrices. This is of particular importance in a random-access, high-throughput MS set up, wherein several different analytes exhibiting different chemical properties have to be measured in a short amount of time.

The present invention relates to a method of determining the presence or the level of an analyte of interest in a sample by a chip based nanoESI detection system which allows a efficiency detection of at least one analyte of interest, e.g. such as steroids, proteins, and other types of analytes, in biological samples.

It is an object of the present invention to provide a method, a diagnostic system, a kit and the use thereof for efficiently detection of an analyte of interest by a chip based nanoESI detection system.

This object is or these objects are solved by the subject matter of the independent claims. Further embodiments are subjected to the dependent claims.

### SUMMARY OF THE INVENTION

In the following, the present invention relates to the following aspects:

In a first aspect, the present invention relates to method of determining the presence or the level of an analyte of interest in a sample by a chip based nanoESI detection system, wherein the chip based nanoESI detection system comprises an electrically conductive pipette tip and a nano-electrospray nozzle, said method comprises the following steps:
a) Providing the sample including the analyte of interest and a matrix, wherein the matrix is non-magnetic,
b) Providing a microparticle, wherein the microparticle is magnetic,
c) Incubation of the microparticle and the analyte of interest to form an analyte-microparticle complex in a sample holder, wherein the analyte-microparticle complex is magnetic,
d) Separating the matrix and the analyte-microparticle complex by magnetic forces,
e) Optionally washing the analyte-microparticle complex in the sample holder,
f) Extracting the analyte from the analyte-microparticle complex by an extraction solvent and magnetic forces, said step (f) comprises
f1) Providing the extraction solvent by the electrically conductive pipette tip,
f2) Contacting the extraction solvent and the analyte-microparticle complex in the sample holder,
f3) Extracting the analyte of interest from the analyte-microparticle complex to form an extracted analyte of interest, wherein the microparticle is retained in the sample holder by magnetic forces during the extracting step f3), wherein the electrically conductive pipette tip comprises the extracted analyte of interest,
g) Directly contacting the electrically conductive pipette tip comprising the extracted analyte of interest and the nano-electrospray nozzle of a chip based nanoESI detection system to form a nano-electrospray for ionization of the extracted analyte of interest. Directly contacting can mean either by directed touching of the respective objects or surfaces or by contact of the liquid phase (e.g. extracted analyte in extraction solvent) with the respective objects or surfaces.
h) Determining the presence or the level of the extracted analyte of interest in the sample using the chip based nanoESI detection system, wherein the chip based nanoESI detection system uses mass spectrometry, ion mobility and/or a combination thereof.

In a second aspect, the present invention relates to the use of the method of the first aspect for determining the presence or the level of an analyte of interest in a sample.

In a third aspect, the present invention relates to a diagnostic system for determining the presence or the level of an analyte of interest in a sample, comprising a chip based nanoESI source, an electrically conductive pipette tip and a detector to carry out the method according to the first aspect, wherein the chip based nanoESI source comprises a nozzle, wherein the detector uses mass spectrometry or ion mobility or combination thereof.

In a fourth aspect, the present invention relates to the use of the diagnostic system of the third aspect in the method of the first aspect.

In a fifth aspect, the present invention relates to a kit suitable to perform a method of the first aspect comprising
(A) a microparticle for enriching or purification the analyte of interest in a sample,
(B) an extraction solvent for extracting the analyte of interest from the microparticle,
(C) optionally an internal standard, and
(D) optionally a catalyst or other reagents. Other reagents are e.g. derivatization reagents.

In a sixth aspect, the present invention relates to the use of a kit of the fifth aspect of the present invention in a method of the first aspect of the present invention.

### LIST OF FIGURES

FIG. 1 shows a method of determining the presence or the level of an analyte of interest in a sample by a chip based nanoESI detection system according to the invention.

FIG. 2 shows a front view of a diagnostic system performing a method according to the invention.

FIG. 3 shows a side view of a diagnostic system performing a method according to the invention.

FIG. 4A shows an analyte enrichment on superparamagnetic beads as microparticle with subsequent extraction and chip based nanoESI detection system from a smooth surface.

FIG. 4B shows an analyte enrichment on superparamagnetic beads as microparticle with subsequent extraction and chip based nanoESI detection system from a smooth surface.

FIG. 5A shows an analyte enrichment on superparamagnetic beads as microparticle with subsequent extraction and chip based nanoESI detection system out of a wellplate.

FIG. 5B shows an analyte enrichment on superparamagnetic beads as microparticle with subsequent extraction and chip based nanoESI detection system out of a wellplate.

FIG. 6 shows the calibration set of Testosterone-13C3 [M+H]⁺, detected after microparticle enrichment, extraction and ionization starting from analyte spiked horse serum in the presence of an internal standard (ISTD) Aldosterone-13C3.

FIG. 7 shows the calibration set of Testosterone-13C3 [M+H]⁺, detected after microparticle enrichment, extraction and ionization starting from analyte spiked horse serum in the presence of an internal standard (ISTD) Aldosterone-13C3.

FIG. 8 shows the calibration set of Phenytoin-13C1-15N2 [M−H]⁻, detected after microparticle enrichment, extraction and ionization starting from analyte spiked horse serum in the presence of an internal standard (ISTD) Aldosterone-13C3.

FIG. 9 shows an extract of FIG. 8 by adding the corresponding enlargement in the range below 500 pg/mL.

FIG. 10 shows an ion mobility separation of different analytes in a mixture, applying the microparticle based sample enrichment with extraction and ionization in the positive ion mode.

FIG. 11A shows a first of five extracted ion mobilograms (0 to 14 ms drift time dt) detected after microparticle enrichment, extraction and ionization out of a single analyte mix.

FIG. 11B shows a second of five extracted ion mobilograms (0 to 14 ms drift time dt) detected after microparticle enrichment, extraction and ionization out of a single analyte mix.

FIG. 11C shows a third of five extracted ion mobilograms (0 to 14 ms drift time dt) detected after microparticle enrichment, extraction and ionization out of a single analyte mix.

FIG. 11D shows a fourth of five extracted ion mobilograms (0 to 14 ms drift time dt) detected after microparticle enrichment, extraction and ionization out of a single analyte mix.

FIG. 11E shows a fifth of five extracted ion mobilograms (0 to 14 ms drift time dt) detected after microparticle enrichment, extraction and ionization out of a single analyte mix.

FIG. 12 shows the comparison of the detection of Testosterone-13C3, applying the microparticle based sample enrichment/purification with extraction and ionization in the positive ion mode, considering a neat solution versus horse serum matrix.

FIG. 13 shows the ion mobility separation of different analytes in a mixture, applying the microparticle based sample enrichment with extraction and ionization in the negative ion mode.

FIG. 14A shows the ion mobility separation of a first analyte in a mixture, applying the microparticle based sample enrichment with extraction and ionization in the negative ion mode.

FIG. 14B shows the ion mobility separation of a second analyte in a mixture, applying the microparticle based sample enrichment with extraction and ionization in the negative ion mode.

FIG. 14C shows the ion mobility separation of a third analyte in a mixture, applying the microparticle based sample enrichment with extraction and ionization in the negative ion mode.

FIG. 14D shows the ion mobility separation of a fourth analyte in a mixture, applying the microparticle based sample enrichment with extraction and ionization in the negative ion mode.

FIG. 14E shows the ion mobility separation of a fifth analyte in a mixture, applying the microparticle based sample enrichment with extraction and ionization in the negative ion mode.

FIG. 15 shows the comparison of the detection of Estradiol-13C3, applying the microparticle based sample enrichment/purification with extraction and ionization in the negative ion mode, considering a neat solution versus horse serum matrix.

FIG. 16 shows the application of microparticle, e.g. magnetic immunobeads, for the detection of Estradiol-13C3, applying the microparticle sample enrichment with extraction and ionization in the negative ion mode.

FIG. 17 shows an application of microparticle, e.g. magnetic immunobeads, for the detection of Testosterone-13C3, applying the microparticle based sample enrichment with extraction and ionization in the positive ion mode.

### DETAILED DESCRIPTION OF THE INVENTION

Before the present invention is described in detail below, it is to be understood that this invention is not limited to the particular embodiments and examples described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps. The term “including” and “comprising” can be used interchangeable.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “4% to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “Mass Spectrometry” (“Mass Spec” or “MS”) or “mass spectrometric determination” or “mass spectrometric analysis” relates to an analytical technology used to identify compounds by their mass. MS is a methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). The term “ionization” or “ionizing” refers to the process of generating an analyte ion having a net charge equal to one or more units. Negative ions are those having a net negative charge of one or more units, while positive ions are those having a net positive charge of one or more units. The MS method may be performed either in “negative ion mode”, wherein negative ions are generated and detected, or in “positive ion mode” wherein positive ions are generated and detected.

“Tandem mass spectrometry” or “MS/MS” involves multiple steps of mass spectrometry selection, wherein fragmentation of the analyte occurs in between the stages. In a tandem mass spectrometer, ions are formed in the ion source and separated by mass-to-charge ratio in the first stage of mass spectrometry (MS1). Ions of a particular mass-to-charge ratio (precursor ions or parent ion) are selected and fragment ions (or daughter ions) are created by collision-induced dissociation, ion-molecule reaction, or photodissociation. The resulting ions are then separated and detected in a second stage of mass spectrometry (MS2).

Since a mass spectrometer separates and detects ions of slightly different masses, it easily distinguishes different isotopes of a given element. Mass spectrometry is thus, an important method for the accurate mass determination and characterization of analytes, including but not limited to low-molecular weight analytes, peptides, polypeptides or proteins. Its applications include the identification of proteins and their post-translational modifications, the elucidation of protein complexes, their subunits and functional interactions, as well as the global measurement of proteins in proteomics. De novo sequencing of peptides or proteins by mass spectrometry can typically be performed without prior knowledge of the amino acid sequence.

The term “electrospray ionization” or “ESI,” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber, which is heated slightly to prevent condensation and to evaporate solvent. As the droplets get smaller the electrical surface charge density increases until such time that the natural repulsion between like charges causes ions as well as neutral molecules to be released.

The term “nano electrospray ionization” or “nanoESI” can refer to a classical 10 oder 20 nL/m electrospray ionization. It can be methods typically using flow rates below 1 L/min either in static or dynamic mode. Preferably, nanoESI uses a flow rate of 10 or 20 nl/min to 500 nl/min, e.g. 500 nl/min. 500 nl/min is equal to 0.5 μl/min.

The term “static nanoESI mass spectrometry” is used in the context of the present disclosure as a non-continuous flow nanoESI option. The analysis is typically defined by a discrete sample being loaded into an emitter, while a nano electrospray is formed during application of voltage together with a constant gaseous backpressure. In contrast, dynamic nanoESI mass spectrometry is characterized by a mobile phase pumped at low flow rates through a small diameter emitter, while applying a voltage.

In the context of the present disclosure, the term “analyte”, “analyte molecule”, or “analyte(s) of interest” are used interchangeably referring the chemical species to be analysed via mass spectrometry, in particular nanoESI mass spectrometry. Chemical species suitable to be analysed via mass spectrometry, i.e. analytes, can be any kind of molecule present in a living organism, include but are not limited to nucleic acid (e.g. DNA, mRNA, miRNA, rRNA etc.), amino acids, peptides, proteins (e.g. cell surface receptor, cytosolic protein etc.), metabolite or hormones (e.g. testosterone, estrogen, estradiol, etc.), fatty acids, lipids, carbohydrates, steroids, ketosteroids, secosteroids (e.g. Vitamin D), molecules characteristic of a certain modification of another molecule (e.g. sugar moieties or phosphoryl residues on proteins, methyl-residues on genomic DNA) or a substance that has been internalized by the organism (e.g. therapeutic drugs, drugs of abuse, toxin, etc.) or a metabolite of such a substance. Such analyte may serve as a biomarker. In the context of present invention, the term “biomarker” refers to a substance within a biological system that is used as an indicator of a biological state of said system.

Analytes may be present in a sample of interest, e.g. a biological or clinical sample. The term “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, spinal fluid, urine, saliva, and lymphatic fluid, or solid samples such as dried blood spots and tissue extracts. Further examples of samples are cell cultures or tissue cultures.

In the context of the present disclosure, the sample may be derived from an “individual” or “subject”. Typically, the subject is a mammal. Mammals include, but are not limited to, domesticated animals (e.g., cows, sheep, cats, dogs, and horses), primates (e.g., humans and non-human primates such as monkeys), rabbits, and rodents (e.g., mice and rats).

The term “serum” as used herein is the clear liquid part of the blood hat can be separated from clotted blood. The term “plasma” as used herein is the clear liquid part of blood which contains the blood cells. Serum differs from plasma, the liquid portion of normal unclotted blood containing the red and white cells and platelets. It is the clot that makes the difference between serum and plasma. The term “whole blood” as used herein contains all components of blood, for examples white and red blood cells, platelets, and plasma.

In this context “level” or “level value” encompasses the absolute amount, the relative amount or concentration as well as any value or parameter which correlates thereto or can be derived therefrom.

The term “determining” the level of the analyte of interest, as used herein refers to the quantification of the analyte of interest, e.g. to determining or measuring the level of the analyte of interest in the pretreated sample. The level of the analyte of interest is determined by nanoESI mass spectrometry.

The term “hemolysis reagent” (HR) refers to reagents which lyse cells present in a sample, in the context of this invention hemolysis reagents in particular refer to reagents which lyse the cell present in a blood sample including but not limited to the erythrocytes present in whole blood samples. A well known hemolysis reagent is water (H₂O). Further examples of hemolysis reagents include but are not limited to deionized water, liquids with high osmolarity (e.g. 8M urea), ionic liquids, and different detergents.

Typically, an “internal standard” (ISTD) is a known amount of a substance which exhibits similar properties as the analyte of interest when subjected to the mass spectrometric detection workflow (i.e. including any pre-treatment, enrichment and actual detection step). Although the ISTD exhibits similar properties as the analyte of interest, it is still clearly distinguishable from the analyte of interest. Exemplified, during an ion mobility separation, the ISTD has about the same drift time, respectively ion mobility, as the analyte of interest from the sample. Thus, both the analyte and the ISTD enter the mass spectrometer at the same time. The ISTD however, exhibits a different molecular mass than the analyte of interest from the sample. This allows a mass spectrometric distinction between ions from the ISTD and ions from the analyte by means of their different mass/charge (m/z) ratios. Both are subject to fragmentation and provide daughter ions. These daughter ions can be distinguished by means of their m/z ratios from each other and from the respective parent ions. Consequently, a separate determination and quantification of the signals from the ISTD and the analyte can be performed. Since the ISTD has been added in known amounts, the signal intensity of the analyte from the sample can be attributed to a specific quantitative amount of the analyte. Thus, the addition of an ISTD allows for a relative comparison of the amount of analyte detected, and enables unambiguous identification and quantification of the analyte(s) of interest present in the sample when the analyte(s) reach the mass spectrometer. Typically, but not necessarily, the ISTD is an isotopically labeled variant (comprising e.g. ²H, ¹³C, or ¹⁵N etc. label) of the analyte of interest.

The term “in vitro method” is used to indicate that the method is performed outside a living organism and preferably on body fluids, isolated tissues, organs or cells.

The term “automatically” or “automated” as used herein is a broad term and is to be given its ordinary and customary meaning to a person of ordinary skill in the art and is not to be limited to a special or customized meaning. The term specifically may refer, without limitation, to a process which is performed completely by means of at least one computer and/or computer network and/or machine, in particular without manual action and/or interaction with a user.

A “kit” is any manufacture (e.g., a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like. In particular, each of the container means comprises one of the separate elements to be used in the method of the first aspect. Kits may further comprise one or more other reagents including but not limited to reaction catalyst. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as a optical storage medium (e.g., a Compact Disc) or directly on a computer or data processing device. Moreover, the kit may, comprise standard amounts for the biomarkers as described elsewhere herein for calibration purposes.

The term “microparticles” as used herein is a broad term and is to be given its ordinary and customary meaning to a person of ordinary skill in the art and is not to be limited to a special or customized meaning. The term specifically may refer, without limitation, to an arbitrary particulate matter of microscopic size. The microparticles may have a mean diameter in the range from 100 nm to 100 μm, specifically from 500 200 nm to 50 μm. The microparticles may also be referred to as beads. The microparticles may be of spherical or globular shape. However, slight derivations from the spherical or globular shape may be feasible. In particular, the microparticles have the at least one surface where the analyte of interest can be attached, e.g. covalenty or Van der Waals forces. The term “surface” as used herein is a broad term and is to be given its ordinary and customary meaning to a person of ordinary skill in the art and is not to be limited to a special or customized meaning. The term specifically may refer, without limitation, to an entirety of areas which delimit an arbitrary body from the outside. Thus, the body may have a plurality of surfaces. Specifically, the microparticles may have a core surrounded by the surface. The surface and the core may comprise different materials. Further, the surface and the core may have different properties. Exemplarily, the core may be magnetic. The surface may be configured for capturing molecules, e.g. a broad range of polar to apolar molecules, when the microparticles are incubated with a sample comprising such molecules. The term microparticle and bead can be used interchangeable.

In particular, the microparticles may be selected from the group consisting of: magnetic microparticles, specifically magnetic microparticles having a magnetic core and a modified surface; silica microparticles, specifically silica microparticles having a silica core and a modified surface; melamine resin microparticles, specifically melamine resin microparticles having a melamine resin core and a modified surface; poly(styrene) based microparticles, specifically poly(styrene) based microparticles having a poly(styrene) core and a modified surface; poly(methyl methacrylate) microparticles, specifically poly(methyl methacrylate) microparticles having a poly(methyl methacrylate) core and a modified surface.

However, also other particles may be feasible. The melamine resin microparticles may have a mean diameter of 500 nm to 20 μm, preferably of 2 μm to 4 μm, most preferably of 3 μm. The poly(styrene) based microparticles may have a mean diameter of 500 nm to 50 μm, prefera-bly of 2 μm to 4 μm, most preferably of 3 μm. The poly(methyl methacrylate) microparticles may have a mean diameter of 500 nm to 50 μm, preferably of 2 μm to 4 μm, most preferably of 3 μm. The modified surface of the magnetic microparticles may be a modified poly(styrene) surface and the magnetic microparticles may have a mean diameter of 5 μm to 50 μm, preferably of 10 μm to 30 μm, most preferably of 20 μm. The modified surface of the magnetic microparticles may be a silica surface and the magnetic microparticles may have a mean diameter of 100 nm to 1000 nm, preferably of 200 nm to 500 nm, most preferably of 300 nm. The modified surface of the silica microparticles may be a cyanopropyl silane functionalized surface and the silica microparticles may have a mean diameter of 5 μm to 100 μm, preferably of 20 μm to 80 μm, most preferably of 40 μm. Also other dimen-sions may be feasible.

The term “chip based nanoESI detection system” as used herein is a broad term and is to be given its ordinary and customary meaning to a person of ordinary skill in the art and is not to be limited to a special or customized meaning.

In particular, chip based nanoESI detection system comprises an electrically conductive pipette tip and a nano-electrospray nozzle.

The term “electrically conductive pipette tip” as used herein is a broad term and is to be given its ordinary and customary meaning to a person of ordinary skill in the art and is not to be limited to a special or customized meaning. The electrically conductive pipette tip can comprise an electrically conductive material that is selected from the group consisting of at least partially graphene, carbon nanotubes, carbon black, carbon fibers, stainless steel, aluminum, titanium, chromium, electrically conductive metals and alloys thereof. The electrically conductive pipette tip can be a disposable electrically conductive pipette tip.

The term “nano-electrospray nozzle” as used herein is a broad term and is to be given its ordinary and customary meaning to a person of ordinary skill in the art and is not to be limited to a special or customized meaning. A nano-electrospray nozzle can be a single- and/or multiple use nozzle with an inner diameter smaller than 1 mm. Nano-electrospray nozzles may be arranged on a consumable chip containing an certain amount of nano-electrospray nozzles. The nano-electrospray nozzle can be a disposal nano-electrospray nozzle.

The term “incubation” as used herein is a broad term and is to be given its ordinary and customary meaning to a person of ordinary skill in the art and is not to be limited to a special or customized meaning. The term specifically may refer, without limitation, to a mixing of at least two substances and/or to an addition of at least one substance to another. Specifically, a solid or particulate matter may be added to and/or mixed with a sample of liquid. Apart from the process of adding and/or mixing, the incubation may further comprise a period of time referred to as incubation time. During the incubation time one of the two substances may be adsorbed on a surface of the other one of the two substances. During the incubation time further conditions, such as temperature and/or other conditions, may be chosen e.g. to favor the desired adsorption. Thus, in step b) the microparticles may be added to the sample and may optionally be mixed with the sample. In step b), the sample may be incubated with the microparticles with an incubation time of 1 s to 60 min, preferably of 1 min to 30 min, most preferably of 3 min to 12 min. However, also other durations may be feasible.

The term “analyte-microparticle-complex” as used herein is a broad term and is to be given its ordinary and customary meaning to a person of ordinary skill in the art and is not to be limited to a special or customized meaning. The term specifically may refer, without limitation, to an ensemble comprising at least one microparticle and at least one analyte, specifically one microparticle and a plurality of analytes. The microparticle and the analyte, specifically the analytes, forming the complex may be reversibly associated. Thus, the components of the complex may, at least under certain conditions, leave the complex or dissociate from the complex. The analyte-microparticle-complex may form on the basis of at least one force of attraction between the microparticle and the analyte. In particular, the force of attraction may act between the surface of the microparticles and the analyte. Thus, the analyte that may initially be distributed in the sample, specifically in a liquid phase of the sample, may accumulate in a process of adsorption at the surface of the microparticles. The forces of attraction may include van der Waals forces and electrostatic attraction. Other forces of attraction are feasible. Specifically, at least one chemical bond may be formed between the microparticle and the analyte, specifically between the surface of the microparticle and the analyte, as part of the formation of the analyte-microparticle-complex. The analyte-microparticle-complex may also be referred to as analyte loaded microparticles.

In step c), the analyte of interest can be incubated with the microparticles whereby the analyte can be adsorbed on the surface of the microparticles and the analyte-microparticle-complex can be formed. In this context, the expression can be understood that a plurality of analyte-microparticle-complexes are formed. This, in step c), the sample may be incubated with the microparticles whereby the analyte can be adsorbed on the surface of the microparticles and the analyte-microparticle-complexes can be formed.

The term “sample holder” as used herein is a broad term and is to be given its ordinary and customary meaning to a person of ordinary skill in the art and is not to be limited to a special or customized meaning. The sample holders intended use can be providing the sample for further analysis. Sample holder options are e.g. well plates, glass plates and plain or structured surfaces.

The term “magnetic forces” as used herein is a broad term and is to be given its ordinary and customary meaning to a person of ordinary skill in the art and is not to be limited to a special or customized meaning. Magnetic forces can result from the application of a magnetic field, introduced by a permanent magnet or an electromagnet.

The term “extracting” as used herein is a broad term and is to be given its ordinary and customary meaning to a person of ordinary skill in the art and is not to be limited to a special or customized meaning. An extraction is a separation process consisting of the separation oa substance, e.g. analyte, from a matrix.

The term “contacting” as used herein is a broad term and is to be given its ordinary and customary meaning to a person of ordinary skill in the art and is not to be limited to a special or customized meaning. Generally, it can be described by coming together or touching, as of objects or surfaces.

The term “directly contacting” as used herein is a broad term and is to be given its ordinary and customary meaning to a person of ordinary skill in the art and is not to be limited to a special or customized meaning. Directly contacting can mean either by directed touching of the respective objects or surfaces or by contact of the liquid phase (e.g. extracted analyte in extraction solvent) with the respective objects or surfaces.

The term “random-access” as used herein is a broad term and is to be given its ordinary and customary meaning to a person of ordinary skill in the art and is not to be limited to a special or customized meaning. In general, the process can describe the ability to perform an analysis or transferring information directly at random rather than being accessed in a fixed sequence.

A “clinical diagnostics system” is a laboratory automated apparatus dedicated to the analysis of samples for in vitro diagnostics. The clinical diagnostics system may have different configurations according to the need and/or according to the desired laboratory workflow. Additional configurations may be obtained by coupling a plurality of apparatuses and/or modules together. A “module” is a work cell, typically smaller in size than the entire clinical diagnostics system, which has a dedicated function. This function can be analytical but can be also pre-analytical or post analytical or it can be an auxiliary function to any of the pre-analytical function, analytical function or post-analytical function. In particular, a module can be configured to cooperate with one or more other modules for carrying out dedicated tasks of a sample processing workflow, e.g. by performing one or more pre-analytical and/or analytical and/or post-analytical steps. In particular, the clinical diagnostics system can comprise one or more analytical apparatuses, designed to execute respective workflows that are optimized for certain types of analysis, e.g. clinical chemistry, immunochemistry, coagulation, hematology, liquid chromatography separation, mass spectrometry, etc. Thus the clinical diagnostic system may comprise one analytical apparatus or a combination of any of such analytical apparatuses with respective workflows, where pre-analytical and/or post analytical modules may be coupled to individual analytical apparatuses or be shared by a plurality of analytical apparatuses. In alternative pre-analytical and/or post-analytical functions may be performed by units integrated in an analytical apparatus. The clinical diagnostics system can comprise functional units such as liquid handling units for pipetting and/or pumping and/or mixing of samples and/or reagents and/or system fluids, and also functional units for sorting, storing, transporting, identifying, separating, detecting.

The clinical diagnostic system can comprise a sample preparation station for the automated preparation of samples comprising analytes of interest, optionally a separation station. In particular, the clinical diagnostic system is free of a separation station, e.g. a LC-HPLC unit or HPLC unit.

The clinical diagnostic system can further comprise a controller programmed to assign samples to pre-defined sample preparation workflows each comprising a pre-defined sequence of sample preparation steps and requiring a pre-defined time for completion depending on the analytes of interest. The clinical diagnostic system can further comprise a mass spectrometer (MS).

A “sample preparation station” can be a pre-analytical module coupled to one or more analytical apparatuses or a unit in an analytical apparatus designed to execute a series of sample processing steps aimed at removing or at least reducing interfering matrix components in a sample and/or enriching analytes of interest in a sample. Such processing steps may include any one or more of the following processing operations carried out on a sample or a plurality of samples, sequentially, in parallel or in a staggered manner: pipetting (aspirating and/or dispensing) fluids, pumping fluids, mixing with reagents, incubating at a certain temperature, heating or cooling, centrifuging, separating, filtering, sieving, drying, washing, resuspending, aliquoting, transferring, storing, etc.).

The clinical diagnostic system, e.g. the sample preparation station, may also comprise a buffer unit for receiving a plurality of samples before a new sample preparation start sequence is initiated, where the samples may be individually randomly accessible and the individual preparation of which may be initiated according to the sample preparation start sequence.

### Embodiments

In a first aspect, the present invention relates to a method of determining the presence or the level of an analyte of interest in a sample by a chip based nanoESI detection system, wherein the chip based nanoESI detection system comprises an electrically conductive pipette tip and a nano-electrospray nozzle, said method comprises the following steps:
a) Providing the sample including the analyte of interest and a matrix, wherein the matrix is non-magnetic,
b) Providing a microparticle, wherein the microparticle is magnetic,
c) Incubation of the microparticle and the analyte of interest to form an analyte-microparticle complex in a sample holder, wherein the analyte-microparticle complex is magnetic,
d) Separating the matrix and the analyte-microparticle complex by magnetic forces,
e) Optionally washing the analyte-microparticle complex in the sample holder,
f) Extracting the analyte from the analyte-microparticle complex by an extraction solvent and magnetic forces, said step (f) comprises
f1) Providing the extraction solvent by the electrically conductive pipette tip,
f2) Contacting the extraction solvent and the analyte-microparticle complex in the sample holder,
f3) Extracting the analyte of interest from the analyte-microparticle complex to form an extracted analyte of interest, wherein the microparticle is retained in the sample holder by magnetic forces during the extracting step f3), wherein the electrically conductive pipette tip comprises the extracted analyte of interest,
g) Directly contacting the electrically conductive pipette tip comprising the extracted analyte of interest and the nano-electrospray nozzle of a chip based nanoESI detection system to form a nano-electrosprayESI spray for ionization of the extracted analyte of interest, and
h) Determining the presence or the level of the extracted analyte of interest in the sample using the chip based nanoESI detection system, wherein the chip based nanoESI detection system uses mass spectrometry, ion mobility and/or a combination thereof.

This method allows that the performance and throughput of e.g. a stand-alone MS can be significantly upgraded by coupling with efficient sample processing strategies. Miniaturization and integration into the final analysis, and full automation of the whole analytical process can enhance throughput and reduce complexity and separately performed sample preparation. The capabilities of direct MS can further be enhanced by highly selective gas-phase separation techniques such as high-resolution MS and ion mobility MS.

The method according to the invention can show the following advantages:

### Reduced Complexity and Robustness

a. extremely low sample/eluent/extraction solution consumption
b. highly efficient extraction
c. significantly less sample injection and therefore less background/matrix in the MS
d. single use tips and spray nozzles
e. modular assembly

### Simplified Workflow

f. sample preparation by microparticle workflow is directly connected to ionization for MS
g. microparticle workflow can simply be adapted to specific analytes of interest without changing the ionization
h. no chromatographic column
i. no HPLC gradient/eluent system
j. isobaric separation by ion mobility or immuno-functionalized microparticles
k. scalable for high- as well as low-throughput

### Performance

l. enhanced sensitivity by nanoESI
m. analyte concentration in microparticle extraction step
n. increased S/N levels
o. spray and multiple MS experiments

In embodiments of the first aspect of the invention, the electrically conductive pipette tip comprising the extracted analyte of interest is free of a microparticle.

In embodiments of the first aspect of the invention, the material of the electrically conductive pipette tip comprises a electrically conductive material selected from the group consisting of at least partially graphene, carbon nanotubes, carbon black, carbon fibers, stainless steel, aluminum, titanium, chromium, electrically conductive metals and alloys thereof. Preferably, the electrically conductive material is selected from the group consisting of graphene, carbon nanotubes, carbon black, carbon fibers and combinations thereof.

In embodiments of the first aspect of the invention, the electrical conductive pipette tip comprises a microparticle content with respect to the total content of the microparticle which is less than 20%, 15%, 10%, 8%, 6%, 4%, 2%, 1%, 0.1% or 0.01%.

In embodiments of the first aspect of the invention, the directly contact between the electrically conductive pipette tip and the nozzle of a chip based nanoESI detection system is a direct mechanical contact.

In embodiments of the first aspect of the invention, the directly contact between the electrically conductive pipette tip and the nozzle of a chip based nanoESI detection system is a direct electrical contact. This can mean that a kind of bridge between the analyte of interest and the nozzle can be formed.

In embodiments of the first aspect of the invention, the directly contact between the electrically conductive pipette tip and the nozzle of a chip based nanoESI detection system is a direct contact between the extracted analyte of interest and the nozzle of a chip based nanoESI detection system.

In embodiments of the first aspect of the invention, the directly contact between the electrically conductive pipette tip and the nozzle of a chip based nanoESI detection system is a direct contact between the extracted analyte of interest and the nozzle of a chip based nanoESI detection system and the electrically conductive pipette tip.

In embodiments of the first aspect of the invention, the ratio microparticle:extraction solvent in step f) is in the range of 0.1:1 to 50:1, preferably 0.5:1 to 25:1, more preferably 1:1 to 10:1. The ratio means mass percentage (w/w).

In embodiments of the first aspect of the invention, the microparticle is supramagnetic or paramagnetic.

In embodiments of the first aspect of the invention, the magnetic forces is induced by a permanent magnet or electrical magnet.

In embodiments of the first aspect of the invention, the sample is a biological sample is derived from an individual, preferably a human being.

In embodiments of the first aspect of the invention, the sample is biological or clinical sample selected from the group consisting of blood, serum, plasma, urine, saliva, spinal fluid, and a dried blood spot.

In embodiments of the first aspect of the invention, the sample is a hemolysed whole-blood sample, particularly a hemolysed human whole-blood sample. The hemolysis can be induced by using a hemolysis reagent.

In embodiments of the first aspect of the invention, the matrix comprises analyte-interfering components derived from biological samples, microparticle, sample preparation solutions, mixtures or combinations thereof.

In embodiments of the first aspect of the invention, the matrix is a solution.

In embodiments of the first aspect of the invention, the matrix comprises an internal standard.

In embodiments of the first aspect of the invention, step e) comprises
e1) Addition of a washing solution, and
e2) Removal of washing supernatant after magnetic separation.

In embodiments of the first aspect of the invention, before step f) and preferably after step e) the method comprises the step:
i) Drying the analyte-microparticle complex, and/or
ii) Storing the analyte-microparticle complex.

In embodiments of the first aspect of the invention, the method is automated.

In embodiments of the first aspect of the invention, method is performed in a random-excess mode.

In embodiments of the first aspect of the invention, the method is an in-vitro diagnostic method.

In embodiments of the first aspect of the invention, the method is performed continuously.

In embodiments of the first aspect of the invention, the analyte of interest is selected from the group consisting of nucleic acid, amino acid, peptide, protein, metabolite, hormones, fatty acid, lipid, carbohydrate, steroid, ketosteroid, secosteroid, a molecule characteristic of a certain modification of another molecule, a substance that has been internalized by the organism, a metabolite of such a substance and combination thereof.

In embodiments of the first aspect of the present invention, the analyte of interest is selected from the group consisting of testosterone, epitestosterone, dihydrotestosterone (DHT), desoxymethyltestosterone (DMT), tetrahydrogestrinone (THG), aldosterone, estrone, 4-hydroxyestrone, 2-methoxyestrone, 2-hydroxyestrone, 16-ketoestradiol, 16-alpha-hydroxyestrone, 2-hydroxyestrone-3-methylether, prednisone, prednisolone, pregnenolone, progesterone, dehydroepiandrosterone (DHEA), 17-hydroxypregnenolone, 17-hydroxyprogesterone, androsterone, epiandrosterone, Δ4-androstenedione, 11-deoxycortisol, corticosterone, 21-deoxycortisol, 11-deoxycorticosterone, allopregnanolone and aldosterone.

In embodiments of the first aspect of the present invention, the analyte of interest is selected from the group consisting of Δ8-tetrahydrocannabinolic acid, benzoylecgonin, salicylic acid, 2-hydroxybenzoic acid, gabapentin, pregabalin, valproic acid, vancomycin, methotrexate, mycophenolic acid, montelukast, repaglinide, furosemide, telmisartan, gemfibrozil, diclofenac, ibuprofen, indomethacin, zomepirac, isoxepac and penicillin. In embodiments of the first aspect of the present invention, the analyte molecule comprising one or more carboxyl groups is an amino acid selected from the group consisting of arginine, lysine, aspartic acid, glutamic acid, glutamine, asparagine, histidine, serine, threonine, tyrosine, cysteine, tryptophan, alanine, isoleucine, leucine, methionine, phenyalanine, valine, proline and glycine.

In embodiments of the first aspect of the present invention, the analyte of interest is selected from the group consisting of pyridoxal, N-acetyl-D-glucosamine, alcaftadine, streptomycin and josamycin.

In embodiments of the first aspect of the present invention, the analyte of interest is selected from the group consisting of cocaine, heroin, Ritalin, aceclofenac, acetylcholine, amcinonide, amiloxate, amylocaine, anileridine, aranidipine artesunate and pethidine.

In embodiments of the first aspect of the present invention, the analyte of interest is selected from the group consisting of cantharidin, succinic anhydride, trimellitic anhydride and maleic anhydride.

In embodiments of the first aspect of the present invention, the analyte of interest is selected from the group consisting of cholecalciferol (vitamin D3), ergocalciferol (vitamin D2), calcifediol, calcitriol, tachysterol, lumisterol and tacalcitol. In particular, the secosteroid is vitamin D, in particular vitamin D2 or D3 or derivates thereof. In particular embodiments, the secosteroid is selected from the group consisting of vitamin D2, vitamin D3, 25-hydroxyvitamin D2, 25-hydroxyvitamin D3 (calcifediol), 3-epi-25-hydroxyvitamin D2, 3-epi-25-hydroxyvitamin D3, 1,25-dihydroxyvitamin D2, 1,25-dihydroxyvitamin D3 (calcitriol), 24,25-dihydroxyvitamin D2, 24,25-dihydroxyvitamin D3. In embodiments of the first aspect of the present invention, the analyte molecule comprising one or more diene groups is selected from the group consisting of vitamin A, tretinoin, isotretinoin, alitretinoin, natamycin, sirolimus, amphotericin B, nystatin, everolimus, temsirolimus and fidaxomicin.

In embodiments of the first aspect of the present invention, the analyte of interest is selected from the group consisting of benzyl alcohol, menthol, L-camitine, pyridoxine, metronidazole, isosorbide mononitrate, guaifenesin, clavulanic acid, Miglitol, zalcitabine, isoprenaline, aciclovir, methocarbamol, tramadol, venlafaxine, atropine, clofedanol, alpha-hydroxyalprazolam, alpha-Hydroxytriazolam, lorazepam, oxazepam, Temazepam, ethyl glucuronide, ethylmorphine, morphine, morphine-3-glucuronide, buprenorphine, codeine, dihydrocodeine, p-hydroxypropoxyphene, O-desmethyltramadol, Desmetramadol, dihydroquinidine and quinidine. In embodiments of the first aspect of the present invention, wherein the analyte molecule comprises more than one hydroxyl groups, the analyte is selected from the group consisting of vitamin C, glucosamine, mannitol, tetrahydrobiopterin, cytarabine, azacitidine, ribavirin, floxuridine, Gemcitabine, Streptozotocin, adenosine, Vidarabine, cladribine, estriol, trifluridine, clofarabine, nadolol, zanamivir, lactulose, adenosine monophosphate, idoxuridine, regadenoson, lincomycin, clindamycin, Canagliflozin, tobramycin, netilmicin, kanamycin, ticagrelor, epirubicin, doxorubicin, arbekacin, streptomycin, ouabain, amikacin, neomycin, framycetin, paromomycin, erythromycin, clarithromycin, azithromycin, vindesine, digitoxin, digoxin, metrizamide, acetyldigitoxin, deslanoside, Fludarabine, clofarabine, gemcitabine, cytarabine, capecitabine, vidarabine, and plicamycin.

In embodiments of the first aspect of the present invention, the analyte of interest is selected from the group consisting of thiomandelic acid, DL-captopril, DL-thiorphan, N-acetylcysteine, D-penicillamine, glutathione, L-cysteine, zofenoprilat, tiopronin, dimercaprol and succimer.

In embodiments of the first aspect of the present invention, the analyte of interest is selected from the group consisting of glutathione disulfide, dipyrithione, selenium sulfide, disulfiram, lipoic acid, L-cystine, fursultiamine, octreotide, desmopressin, vapreotide, terlipressin, linaclotide and peginesatide. Selenium sulfide can be selenium disulfide, SeS₂, or selenium hexasulfide, Se₂S₆.

In embodiments of the first aspect of the present invention, the analyte of interest is selected from the group consisting of carbamazepine-10,11-epoxide, carfilzomib, furosemide epoxide, fosfomycin, sevelamer hydrochloride, cerulenin, scopolamine, tiotropium, tiotropium bromide, methylscopolamine bromide, eplerenone, mupirocin, natamycin, and troleandomycin.

In embodiments of the first aspect of the present invention, the analyte of interest is selected from the group consisting of estrogen, estrogen-like compounds, estrone (E1), estradiol (E2), 17a-estradiol, 17b-estradiol, estriol (E3), 16-epiestriol, 17-epiestriol, and 16, 17-epiestriol and/or metabolites thereof. In embodiments, the metabolites are selected from the group consisting of estriol, 16-epiestriol (16-epiE3), 17-epiestriol (17-epiE3), 16,17-epiestriol (16,17-epiE3), 16-ketoestradiol (16-ketoE2), 16a-hydroxyestrone (16a-OHE1), 2-methoxyestrone (2-MeOE1), 4-methoxyestrone (4-MeOE1), 2-hydroxyestrone-3-methyl ether (3-MeOE1), 2-methoxyestradiol (2-MeOE2), 4-methoxyestradiol (4-MeOE2), 2-hydroxyestrone (2-OHE1), 4-hydroxyestrone (4-OHE1), 2-hydroxyestradiol (2-OHE2), estrone (E1), estrone sulfate (E1s), 17a-estradiol (E2a), 17b-estradiol (E2B), estradiol sulfate (E2S), equilin (EQ), 17a-dihydroequilin (EQa), 17b-dihydroequilin (EQb), Equilenin (EN), 17-dihydroequilenin (ENa), 17α-dihydroequilenin, 17β-dihydroequilenin (ENb), Δ8,9-dehydroestrone (dEl), Δ8,9-dehydroestrone sulfate (dEls), Δ9-tetrahydrocannabinol and mycophenolic acid. β or b can be used interchangeable. α and a can be used interchangeable.

In embodiments of the first aspect of the present invention, the analyte of interest is selected from the group consisting of 3,4-methylenedioxyamphetamine, 3,4-methylenedioxy-N-ethylamphetamine, 3,4-methylenedioxymethamphetamine, Amphetamine, Methamphetamine, N-methyl-1,3-benzodioxolylbutanamine, 7-aminoclonazepam, 7-aminoflunitrazepam, 3,4-dimethylmethcathinone, 3-fluoromethcathinone, 4-methoxymethcathinone, 4-methylethcathinone, 4-methylmethcathinone, amfepramone, butylone, ethcathinone, elephedrone, methcathinone, methylone, methylenedioxypyrovalerone, benzoylecgonine, dehydronorketamine, ketamine, norketamine, methadone, normethadone, 6-acetylmorphine, diacetylmorphine, morphine, norhydrocodone, oxycodone, oxymorphone, phencyclidine, norpropoxyphene, amitriptyline, clomipramine, dothiepin, doxepin, imipramine, nortriptyline, trimipramine, fentanyl, glycylxylidide, lidocaine, monoethylglycylxylidide, N-acetylprocainamide, procainamide, pregabalin, 2-Methylamino-1-(3,4-methylendioxyphenyl)butan, N-methyl-1,3-benzodioxolylbutanamine, 2-Amino-1-(3,4-methylendioxyphenyl)butan, 1,3-benzodioxolylbutanamine, normeperidine, O-Destramadol, desmetramadol, tramadol, lamotrigine, Theophylline, amikacin, gentamicin, tobramycin, vancomycin, Methotrexate, Gabapentin sisomicin and 5-methylcytosine.

In embodiments of the first aspect of the present invention, the analyte of interest is selected from the group consisting of ribose, desoxyribose, arabinose, ribulose, glucose, mannose, galactose, fucose, fructose, N-acetylglucosamine, N-acetylgalactosamine, neuraminic acid and N-acetylneurominic acid. In embodiments, the analyte molecule is an oligosaccharide, in particular selected from the group consisting of a disaccharide, trisaccharid, tetrasaccharide and polysaccharide. In embodiments of the first aspect of the present invention, the disaccharide is selected from the group consisting of sucrose, maltose and lactose. In embodiments of the first aspect of the present invention, the analyte molecule is a substance comprising above described mono-, di-, tri-, tetra-, oligo- or polysaccharide moiety.

In embodiments of the first aspect of the present invention, the analyte of interest is zidovudine or azidocillin.

In embodiments of the first aspect of the invention, the method is free of a chromatographic step comprises at least one or more methods selected from the following group: chromatography, high performance liquid chromatography (HPLC), liquid chromatography high performance liquid chromatography (LC-HPLC), gas chromatography (GC), gel permeation chromatography (GPC), flash chromatography. Chromatography is, for example, size exclusion chromatography.

In embodiments of the first aspect of the invention, the methods is performed in the order: a, then b, then c, then d, then optionally e, then f, then g and then h.

In a second aspect, the present invention relates to the use of the method of the first aspect for determining the presence or the level of an analyte of interest in a sample. All embodiments mentioned for the first aspect of the invention apply for the second aspect of the invention and vice versa.

In a third aspect, the present invention relates to a diagnostic system for determining the presence or the level of an analyte of interest in a sample, comprising a chip based nanoESI source, an electrically conductive pipette tip and a detector to carry out the method according to first aspect, wherein the chip based nanoESI source comprises an electrically conductive pipette tip and a nozzle, wherein the detector uses mass spectrometry or ion mobility or combination thereof. All embodiments mentioned for the first aspect of the invention and/or second aspect of the invention apply for the third aspect of the invention and vice versa.

In embodiments of the third aspect of the present invention, the system is a standalone system.

In embodiments of the third aspect of the present invention, the system is integrated in other systems which are able to determine the presence or the level of an analyte of interest based on (electro)chemoluminesence or clinical chemistry.

In embodiments of the third aspect of the present invention, diagnostic system is a clinical diagnostic system.

In embodiments of the third aspect of the present invention, the nanoESI source can be e.g. a chip-based electrospray ionization technology from company Advion. It combines the benefits of liquid chromatography, mass spectrometry, chip-based infusion, fraction collection, and direct surface analysis into one integrated ion source platform. Other known nanoESI sources are also possible. The nanoESI source is known for a skilled person and therefore not explained in detail.

In embodiments of the third aspect of the present invention, the mass spectrometer can be e.g. a triple quadrupole mass spectrometer or a linear ion trap mass spectrometer. A mass spectrometer is known for a skilled person and thus not explained in detail.

In a fourth aspect, the present invention relates to the use of the diagnostic system of third aspect in the method of the first aspect. All embodiments mentioned for the first aspect of the invention and/or second aspect of the invention and/or third aspect of the invention apply for the fourth aspect of the invention and vice versa.

In a fifth aspect, the present invention relates to a kit suitable to perform a method of any one of the preceding aspects comprising
(A) a microparticle for enriching or purification the analyte of interest in a sample,
(B) an extraction solvent for extracting the analyte of interest from the microparticle,
(C) optionally an internal standard, and
(D) optionally a catalyst or other reagents, e.g. derivatization reagents. All embodiments mentioned for the first aspect of the invention and/or second aspect of the invention and/or third aspect of the invention and/or fourth aspect of the invention apply for the fifth aspect of the invention and vice versa.

In a sixth aspect, the present invention relates to a the use of a kit of the fifth aspect in a method of the first aspect. All embodiments mentioned for the first aspect of the invention and/or second aspect of the invention and/or third aspect of the invention and/or fourth aspect of the invention and/or fifth aspect of the invention apply for the sixth aspect of the invention and vice versa.

Summarizing and without excluding further possible embodiments, the following embodiments may be envisaged:

Embodiment 1. A method of determining the presence or the level of an analyte of interest in a sample by a chip based nanoESI detection system, wherein the chip based nanoESI detection system comprises an electrically conductive pipette tip and a nano-electrospray nozzle, said method comprises the following steps:
a) Providing the sample including the analyte of interest and a matrix, wherein the matrix is non-magnetic,
b) Providing a microparticle, wherein the microparticle is magnetic,
c) Incubation of the microparticle and the analyte of interest to form an analyte-microparticle complex in a sample holder, wherein the analyte-microparticle complex is magnetic,
d) Separating the matrix and the analyte-microparticle complex by magnetic forces,
e) Optionally washing the analyte-microparticle complex in the sample holder,
f) Extracting the analyte from the analyte-microparticle complex by an extraction solvent and magnetic forces, said step (f) comprises
f1) Providing the extraction solvent by the electrically conductive pipette tip,
f2) Contacting the extraction solvent and the analyte-microparticle complex in the sample holder,
f3) Extracting the analyte of interest from the analyte-microparticle complex to form an extracted analyte of interest, wherein the microparticle is retained in the sample holder by magnetic forces during the extracting step f3), wherein the electrically conductive pipette tip comprises the extracted analyte of interest,
g) Directly contacting the electrically conductive pipette tip comprising the extracted analyte of interest and the nano-electrospray nozzle of a chip based nanoESI detection system to form a nano-electrospray for ionization of the extracted analyte of interest. Directly contacting can mean either by directed touching of the respective objects or surfaces or by contact of the liquid phase (e.g. extracted analyte in extraction solvent) with the respective objects or surfaces.
h) Determining the presence or the level of the extracted analyte of interest in the sample using the chip based nanoESI detection system, wherein the chip based nanoESI detection system uses mass spectrometry, ion mobility and/or a combination thereof.

Embodiment 2. The method of aspect 1, wherein the electrically conductive pipette tip comprising the extracted analyte of interest is free of a microparticle.

Embodiment 3. The method of any of the preceding aspects, wherein the material of the electrically conductive pipette tip comprises a electrically conductive material selected from the group consisting of at least partially graphene, carbon nanotubes, carbon black, carbon fibers, stainless steel, aluminum, titanium, chromium, electrically conductive metals and alloys thereof.

Embodiment 4. The method of any of the preceding aspects, wherein the electrical conductive pipette tip comprises a microparticle content with respect to the total content of the microparticle which is less than 20%, 15%, 10%, 8%, 6%, 4%, 2%, 1%, 0.1% or 0.01%.

Embodiment 5. The method of any of the preceding aspects, wherein the directly contact between the electrically conductive pipette tip and the nozzle of a chip based nanoESI detection system is a direct mechanical contact.

Embodiment 6. The method of any of the preceding aspects, wherein the directly contact between the electrically conductive pipette tip and the nozzle of a chip based nanoESI detection system is a direct electrical contact.

Embodiment 7. The method of any of the preceding aspects, wherein the directly contact between the electrically conductive pipette tip and the nozzle of a chip based nanoESI detection system is a direct contact between the extracted analyte of interest and the nozzle of a chip based nanoESI detection system.

Embodiment 8. The method of any of the preceding aspects, wherein the directly contact between the electrically conductive pipette tip and the nozzle of a chip based nanoESI detection system is a direct contact between the extracted analyte of interest and the nozzle of a chip based nanoESI detection system and the electrically conductive pipette tip.

Embodiment 9. The method of any of the preceding aspects, wherein the ratio microparticle:extraction solvent in step f) is in the range of 0.1:1 to 20:1, preferably 0.5:1 to 15:1, more preferably 1:1 to 1:10 or the ratio microparticle:extraction solvent in step f) is in the range of 0.1:1 to 50:1, preferably 0.5:1 to 25:1, more preferably 1:1 to 10:1. The ratio means mass percentage (w/w).

Embodiment 10. The method of any of the preceding aspects, wherein the microparticle is supramagnetic or paramagnetic.

Embodiment 11. The method of any of the preceding aspects, wherein the magnetic forces is induced by a permanent magnet or electrical magnet.

Embodiment 12. The method of any of the preceding aspects, wherein the sample is a biological sample is derived from an individual, preferably a human being.

Embodiment 13. The method of any of the preceding aspects, wherein the sample is biological or clinical sample selected from the group consisting of blood, serum, plasma, urine, saliva, spinal fluid, and a dried blood spot.

Embodiment 14. The method of any of the preceding aspects, wherein the sample is a hemolysed whole-blood sample, particularly a hemolysed human whole-blood sample.

15. The method of any of the preceding aspects, wherein the matrix comprises analyte-interfering components derived from biological samples, microparticle, sample preparation solutions, mixtures or combinations thereof.

Embodiment 16. The method of any of the preceding aspects, wherein the matrix is a solution.

Embodiment 17. The method of any of the preceding aspects, wherein the matrix comprises an internal standard.

Embodiment 18. The method of any of the preceding aspects, wherein step e) comprises
e1) Addition of a washing solution, and
e2) Removal of washing supernatant after magnetic separation.

Embodiment 19. The method of any of the preceding aspects, wherein before step f) and preferably after step e) the method comprises the step:
i) Drying the analyte-microparticle complex, and/or
ii) Storing the analyte-microparticle complex.

Embodiment 20. The method of any of the preceding aspects, wherein the method is automated.

Embodiment 21. The method of any of the preceding aspects, wherein method is performed in a random-excess mode.

Embodiment 22. The method of any of the preceding aspects, wherein the method is an in-vitro diagnostic method.

Embodiment 23. The method of any of the preceding aspects, wherein the method is performed continuously.

Embodiment 24. The method of any of the preceding aspects, wherein the analyte of interest is selected from the group consisting of nucleic acid, amino acid, peptide, protein, metabolite, hormones, fatty acid, lipid, carbohydrate, steroid, ketosteroid, secosteroid, a molecule characteristic of a certain modification of another molecule, a substance that has been internalized by the organism, a metabolite of such a substance and combination thereof.

Embodiment 25. The method of any of the preceding aspects, wherein the method is free of a chromatographic step comprises at least one or more methods selected from the following group: chromatography, high performance liquid chromatography (HPLC), liquid chromatography high performance liquid chromatography (LC-HPLC), gas chromatography (GC), gel permeation chromatography (GPC), flash chromatography. Chromatography is, for example, size exclusion chromatography.

Embodiment 26. The method of any of the preceding aspects, wherein the methods is performed in the order: a, then b, then c, then d, then optionally e, then f, then g and then h.

Embodiment 27. Use of the method of any one of the preceding aspects 1 to 26 for determining the presence or the level of an analyte of interest in a sample.

Embodiment 28. A diagnostic system for determining the presence or the level of an analyte of interest in a sample, comprising a chip based nanoESI source, an electrically conductive pipette tip and a detector to carry out the method according to any one of preceding aspects, wherein the chip based nanoESI source comprises a nozzle, wherein the detector uses mass spectrometry or ion mobility or combination thereof.

Embodiment 29. The diagnostic system of the preceding aspect 28, wherein the system is a standalone system.

Embodiment 30. The diagnostic system of any of the preceding aspects 28 to 29, wherein the system is integrated in other systems which are able to determine the presence or the level of an analyte of interest based on (electro)chemoluminesence or clinical chemistry.

Embodiment 31. Use of the diagnostic system of the preceding aspects 28 to 30 in the method of any one of the preceding aspects 1 to 26.

Embodiment 32. A kit suitable to perform a method of any one of the preceding aspects 1 to 26 comprising
(A) a microparticle for enriching or purification the analyte of interest in a sample,
(B) an extraction solvent for extracting the analyte of interest from the microparticle,
(C) optionally an internal standard, and
(D) optionally a catalyst or other reagents, e.g. derivatization reagents.

Embodiment 33. Use of a kit of the preceding aspect 32 in a method of any one of the preceding aspects 1 to 26.

### Examples

The following examples are provided to illustrate, but not to limit the presently claimed invention.

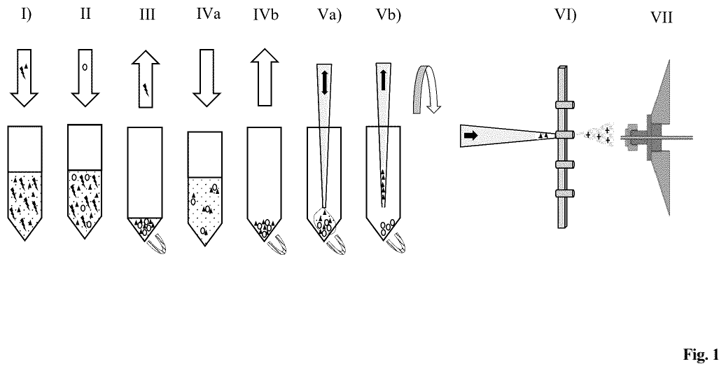

FIG. 1 shows a method of determining the presence or the level of an analyte of interest in a sample by a chip based nanoESI detection system according to the invention. A solid supported bead purification/enrichment workflow in combination with direct extraction and chip based nano-ESI ionization is shown.

In I) of FIG. 1, the sample including the analyte of interest and a matrix is provide. The matrix is non-magnetic. The matrix can comprise analyte-interfering components derived from biological samples, microparticle, sample preparation solutions and/or mixtures. Optionally, the matrix comprises an internal standard.

In II) of FIG. 1, the addition of the a microparticle is shown that are magnetic. Microparticle can be a microparticle suspension. Then, the microparticle and the analyte of interest are incubated to form an analyte-microparticle complex, e.g. in a sample holder. The analyte-microparticle complex is magnetic.

In III) of FIG. 1, supernatant matrix is removed after magnetic separation. The matrix and the analyte-microparticle complex are separated by magnetic forces.

IVa) and IVb) of FIG. 1 shows optionally the washing step of the analyte-microparticle complex, e.g. in the sample holder. IVa) of FIG. 1 shows the addition of a washing solution and IVb) of FIG. 1 shows the removal of washing supernatant after magnetic separation.

Following the extraction procedure in Va) and Vb), an extraction solvent is provided by the electrically conductive pipette tip and the extraction solvent and the analyte-microparticle complex are contacted in the sample holder (Va). The analyte of interest is extracted from the analyte-microparticle complex to form an extracted analyte of interest. In Vb) of FIG. 1, the extracted analyte of interest can be taken up after magnetic separation. Optionally during step Va), the magnet could also be detached, while adding the extraction solvent, what could facilitate a mechanical agitation.

As shown in VI) of FIG. 1, a directly contacting of the electrically conductive pipette tip comprising the extracted analyte of interest and the nano-electrospray nozzle of a chip based nanoESI detection system is shown to form a nano-electrosprayESI spray for ionization of the extracted analyte of interest.

After direct contact VI) to the nozzle of a nano-ESI system and application of a voltage, a continuous nan-ESI spray can be formed. The hereby produced ionic species of the analytes of interest can be analyzed with an analyzing system VII), considering a mass spectrometric device, an ion mobility separation device or combination thereof.

Optionally, between step IVb) and Va), the analyte-microparticle complex can be dried and stored prior to extraction and analysis, what is considered as additional benefit of this method.

Especially in the presence of biological matrices, like blood or serum, a defined enrichment and concentration of desired analytes can enable a direct ionization without using expensive chromatographic systems or fluidic devices. A direct coupling of sample preparation, analyte extraction and ionization has the advantage of reducing large amounts of solvents, fluidic components and disposable materials. Beginning with the sample addition until the final extraction, immediately before ionization, all magnetic bead based analyte workflow steps can be performed in one single assay cup.

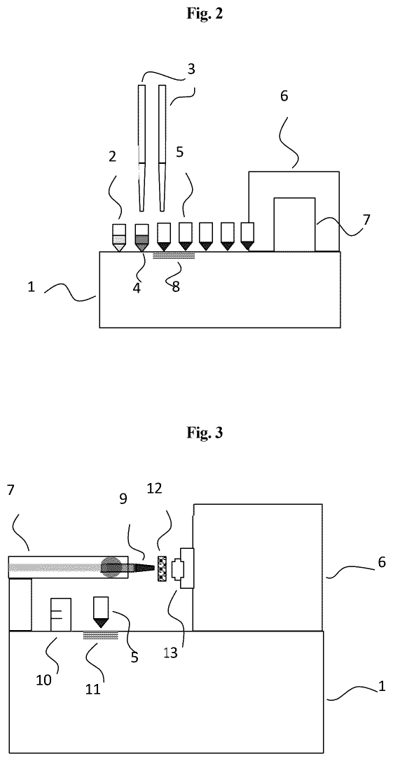

FIGS. 2 and 3 show a front view (FIG. 2) and side view (FIG. 3) of a diagnostic system performing a method according to the invention. The diagnostic system comprises an analysis module 1, a sample solution provided in assay cups 2; pipetting units 3, an analyte solution with microparticle suspension 4, residual analyte adsorbed on the microparticle 5, analysis module 6, microparticle extraction module 7, microparticle separation module 8; electrically conductive pipette tips 9 (microparticle extraction tips), microparticle extraction solvent reservoir 10, microparticle capturing plate 11, nanospray chip (chip based nanoESI source) 12; analysis module inlet 13. A nozzle and detector are not shown in FIGS. 2 and 3.

In order to show the broadness of applicability using the herein described method, different analytes as well as microparticles were tested. Additionally, the use of this method starting from a biological matrix was examined in more detailed. The combination of this sample preparation and ionization method together with ion mobility was demonstrated in the enrichment and separation of multiple analytes in mixtures. Moreover, the capability to quantify analytes of interest was shown successfully.

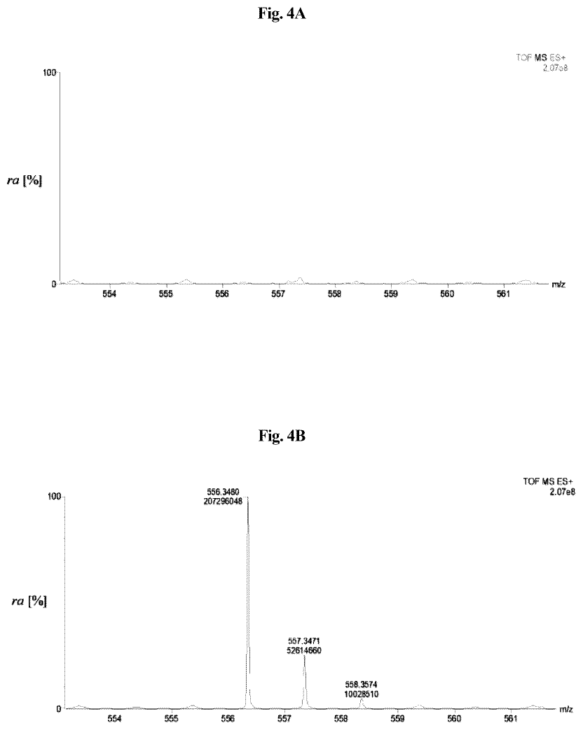

FIGS. 4A and 4B show an analyte enrichment on superparamagnetic beads as microparticle with subsequent extraction and chip based nanoESI detection system from a smooth surface. The respective enlargement of the detected full scan mass spectra (mass-to-charge-ratio m/z553 to 562) around the protonated analyte Leucine enkephalin signal [M+H]+ at m/z 556.3 is displayed (b1)) and can be compared to an extraction of a blank bead sample (a1))—both normalized to the same number of counts (relative abundance ra). The microparticle workflow was started from 100 ng/mL Leucine enkephalin (100 μL), that was provided in a single well of a wellplate (Twin.tech® PCR plate 96, skirted, 150 μL volume, Eppendorf AG), while in a different well 100 μL blank deionized water was put for comparison. 15 μL of a microparticle suspension (10 mg/mL, BeadA=superparamagnetic polystyrene coated carboxylic acid modified bead) was added to both samples and incubated for 3 min in total. After magnetic separation and removal of the supernatant, the analyte-microparticle complexes were washed 2× with deionized water (100 L). The residual analyte-microparticle complexes were transferred with a pipette tip on a microscopic glass slide. Following, the analyte was extracted from the microparticles, laying on the glass slide, with an extraction solvent (10 L) containing 80% acetonitrile (ACN)+0.1% formic acid (FA) and subsequently ionized by nanoESI with a chip based instrument (Triversa NanoMate, Advion Inc.). The mass analysis of the resulting ions was performed with a Synapt G2-Si mass spectrometer (Waters Corp.) in the time of flight (ToF) positive ion mode with an acquisition time of 60 s. The analyte enrichment, extraction and subsequent ionization of Leucine enkephalin in combination with microparticle from a microscopic slide was successful, as the protonated analyte ion was clearly detected in FIG. 4B. The blank experiment in FIG. 4A showed only small background signals, proving the capability of selectively analyzing the peptide Leucine enkephalin without a significant interference of background signals directly from a smooth surface.

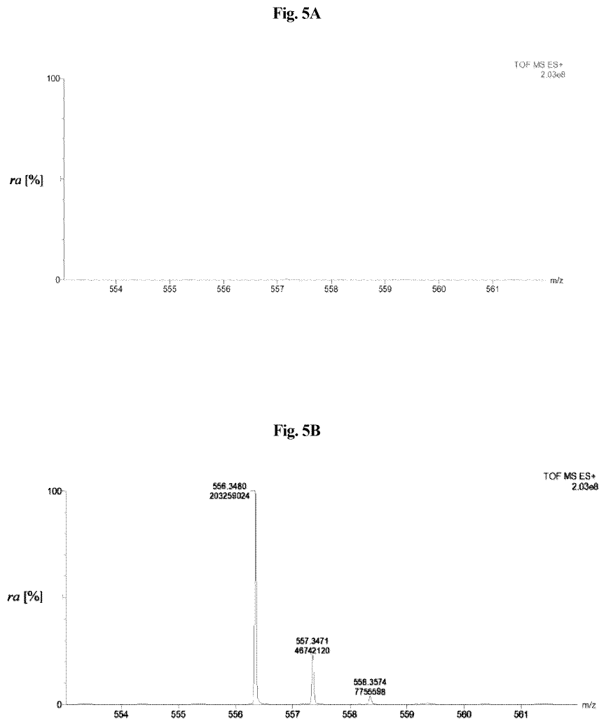

FIGS. 5A and 5B show an analyte enrichment on microparticle, e.g. superparamagnetic beads, with subsequent extraction and chip based nano-ESI detection system out of a wellplate. The respective enlargement of the detected full scan mass spectra (m/z 553 to 562) around the protonated analyte Leucine enkephalin signal [M+H]+ at m/z 556.3 is displayed (b2)) and can be compared to an extraction of a blank bead sample (a2))—both normalized to the same number of counts. The workflow was started from 100 ng/mL Leucine enkephalin (100 μL), that was provided in a single well of a wellplate (Twin.tech® PCR plate 96, skirted, 150 μL volume, Eppendorf AG), while in a different well 100 μL blank deionized water was put for comparison. 15 μL of a microparticle suspension (10 mg/mL, BeadA=superparamagnetic polystyrene coated carboxylic acid modified bead(s)) was added to both samples and incubated for 3 min in total. After magnetic separation and removal of the supernatant, the analyte-microparticle complexes were washed 2× with deionized water (100 μL). The residual analyte-microparticle complexes were then extracted with 80% ACN+0.1% FA (10 μL) and subsequently ionized by nanoESI with a chip based instrument (Triversa NanoMate, Advion Inc.). The mass analysis of the resulting ions was performed with a Synapt G2-Si mass spectrometer (Waters Corp.) in the ToF positive ion mode with an acquisition time of 60 s. The analyte enrichment, extraction and subsequent ionization of Leucine enkephalin in combination with microparticle was successful, as the protonated analyte ion was clearly detected in FIG. 5A. The blank experiment showed no significant overlap, proving the capability of selectively analyzing the peptide Leucine enkephalin directly out of sample wells without an interference of background signals.

This highlights the great potential of combining microparticle based sample preparation techniques with subsequent extraction and chip based nano-ESI ionization directly out of one sample well. After adsorbing and enriching the desired analytes on microparticles, a further processing or transfer in different analyzing vessels is not necessary anymore, as the analyte can directly be extracted and ionized with a single pipetting tip. Therefore, additional materials and time consuming steps can be avoided.

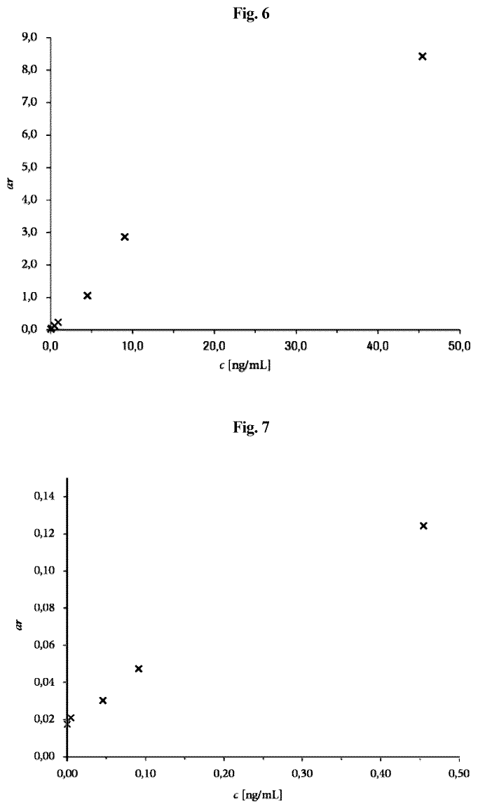

FIGS. 6 and 7 show the calibration set of Testosterone-13C3 [M+H]⁺, detected after microparticle enrichment, extraction and ionization starting from analyte spiked horse serum in the presence of an internal standard (ISTD) Aldosterone-13C3. The following abbreviations are uses: ar=area ratio (=Area(analyte)/Area(ISTD)); c=concentration).

The horizontal axis represents the spiked concentration (c in ng/mL) of Testosterone-13C3 in horse serum (100 L) prior to bead workflow sample preparation. The vertical axis represents the area ratio, derived from Testosterone-13C3 multiple reaction monitoring (MRM) transition m/z 292.1→100.0 (collision energy 18 eV) normalized to the internal standard Aldosterone-13C3 (m/z 364.2→346.0, collision energy 16 eV) over a measurement time of 60 s. FIG. 7 shows an extract of FIG. 6 by adding the corresponding enlargement in the range below 0.5 ng/mL.

As concentration range, eight different samples of Testosterone-13C3 between 46 ng/mL to 4.6 pg/mL were prepared, together with a blank horse serum sample. Every sample contained the same concentration of Aldosterone-13C3 (18 ng/mL), performing as internal standard. The sample preparation included the addition of 15 μL microparticle suspension (10 mg/mL, BeadA) to every sample in a wellplate, incubating them for 3 min in total. After magnetic separation and removal of the supernatant, the analyte-microparticle complexes were washed 2× with water (100 μL). The residual analyte-microparticle complexes were then extracted with 80% ACN+0.1% FA (10 L) and subsequently ionized by nanoESI with a chip based instrument (Triversa NanoMate, Advion Inc.). The mass analysis of the resulting ions was performed with a Xevo TQ-XS mass spectrometer (Waters Corp.) in the positive ion mode. The total analysis time was set to 60 s, choosing the analyte Testosterone-13C3 MRM transition m/z 292.1→100.0 (collision energy 18 eV) and referencing to the Aldosterone-13C3 MRM transition (m/z 364.2→346.0, collision energy 16 eV). A calculation of the detected area ratio was supported, using the TargetLynx software tool (Waters Corp.). Considering the data from this single dilution series, the lowest possible detection was estimated to be around 35 pg/mL.

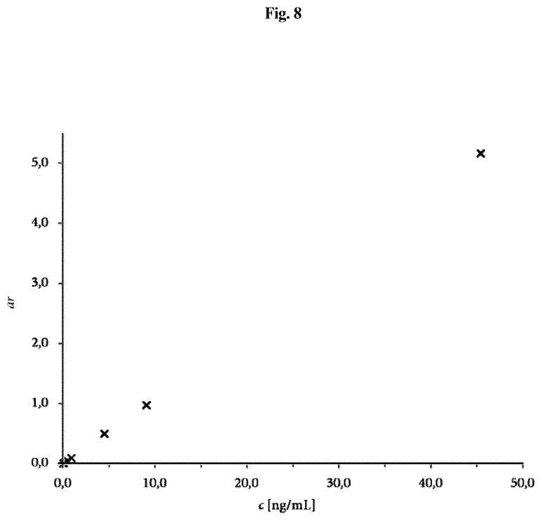

FIG. 8 shows the calibration set of Phenytoin-13C1-15N2 [M−H]⁻, detected after bead enrichment, extraction and ionization starting from analyte spiked horse serum in the presence of an internal standard (ISTD) Aldosterone-13C3.

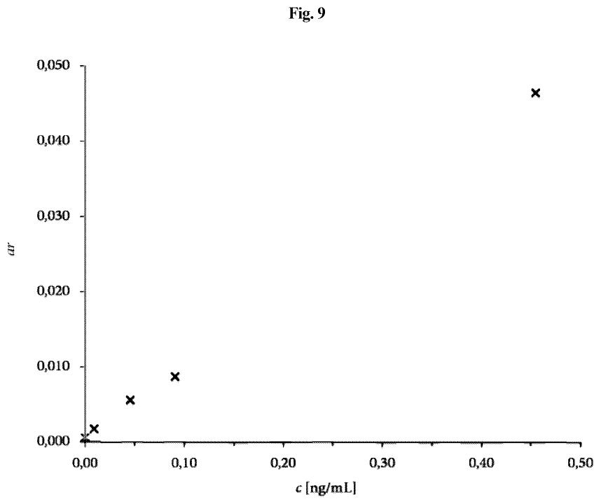

The horizontal axis represents the spiked concentration (c in ng/mL) of Phenytoin-13C1-15N2 in horse serum (100 μL) prior to microparticle workflow sample preparation. The vertical axis represents the area ratio, derived from Phenytoin-13C1-15N2 MRM transition m/z 254.0→103.0 (collision energy 20 eV)normalized to the internal standard Aldosterone-13C3 (m/z 362.2→334.2, collision energy 16 eV) over a measurement time of 60 s. FIG. 9 shows an extract of FIG. 8 by adding the corresponding enlargement in the range below 0.5 ng/mL.

As concentration range, eight different samples of Phenytoin-13C1-15N2 between 46 ng/mL to 4.6 pg/mL were prepared, together with a blank horse serum sample. Every sample contained the same concentration of Aldosterone-13C3 (18 ng/mL), performing as internal standard. The sample preparation included the addition of 15 μL BeadA suspension (10 mg/mL) to every sample in a wellplate, incubating them for 3 min in total. After magnetic separation and removal of the supernatant, the analyte-microparticle complexes were washed 2× with water (100 μL). The residual analyte-microparticle complexes were then extracted with 80% ACN+0.08 mM NH₄F+NH₄OH pH=9.0 (10 μL) and subsequently ionized by nanoESI with a chip based instrument (Triversa NanoMate, Advion Inc.). The mass analysis of the resulting ions was performed with a Xevo TQ-XS mass spectrometer (Waters Corp.) in the negative ion mode. The total analysis time was set to 60 s, choosing the analyte Phenytoin-13C1-15N2 MRM transition m/z 254.0→103.0 (collision energy 20 eV) and referencing to the Aldosterone-13C3 MRM transition (m/z 362.2→334.2, collision energy 16 eV). A calculation of the detected area ratio was supported, using the TargetLynx software tool (Waters Corp.). Considering the data from this single dilution series, the lowest possible detection was estimated to be around 12 pg/mL.

Both results, regarding the dilution series of Testosterone-13C3 and Phenytoin-13C1-15N2, highlight the high sensitivity of this method in the negative as well as positive ion mode, even in the presence of challenging biological matrices.

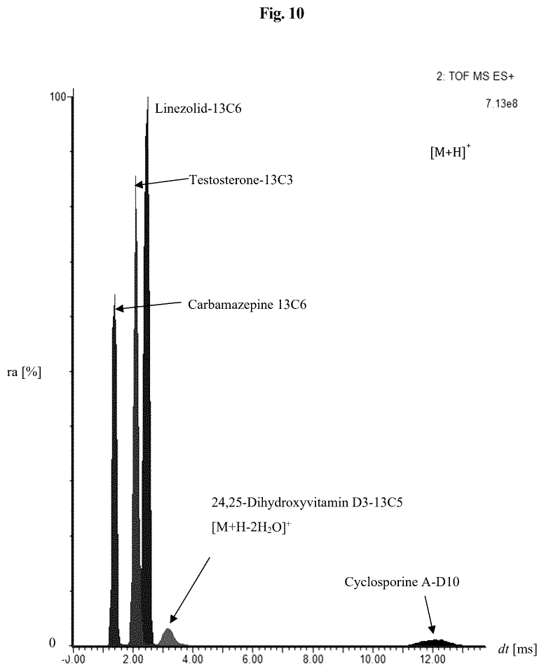

FIG. 10 shows an ion mobility separation of different analytes in a mixture, applying the microparticle based sample enrichment with extraction and ionization in the positive ion mode.

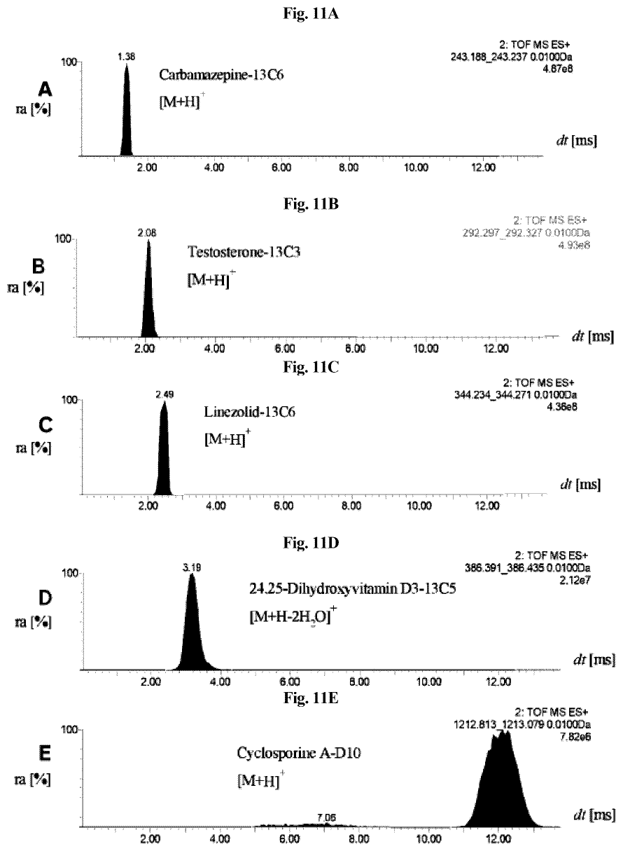

FIGS. 11A to 11E show the overlay of five extracted ion mobilograms (0 to 14 ms drift time dt) detected after microparticle enrichment, extraction and ionization out of a single analyte mix. All five substances represent important diagnostic and therapeutic analytes, representing Carbamazepine-13C6, Testosterone-13C3, Linezolid-13C6, 24,25-Dihydroxyvitamin D3-13C5 and Cyclosporine A-D10 and are spiked to final concentrations of 100 ng/mL (100 μL, neat solution). To the analyte-mix solution in wellplate, 15 μL BeadA suspension (10 mg/mL) was added and incubated for 3 min. After magnetic separation and removal of the supernatant, the analyte-microparticle complex was washed 2× with water (100 μL). The residual analyte-microparticle complex was then extracted with 80% ACN+0.1% FA (10 μL) and subsequently ionized by nanoESI with a chip based instrument (Triversa NanoMate, Advion Inc.). The separation and mass analysis of the resulting ions was performed with a Synapt G2-Si mass spectrometer (Waters Corp.) in combination with ion mobility separation (IMS; wave velocity 650 m/s, wave height 40 V) in the IMS-ToF positive ion mode with an acquisition time of 60 s. All spiked analytes were enriched and extracted successfully out of one single analyte mix. For 24,25-Dihydroxyvitamin D3-13C5, the [M+H−2H₂O]⁺ ion species was observed at m/z 386.2, while all other analytes appeared in their corresponding protonated adducts [M+H]⁺.

Comparison of the detection of Testosterone-13C3, applying the bead based sample enrichment/purification with extraction and ionization in the positive ion mode, considering a neat solution versus horse serum matrix:

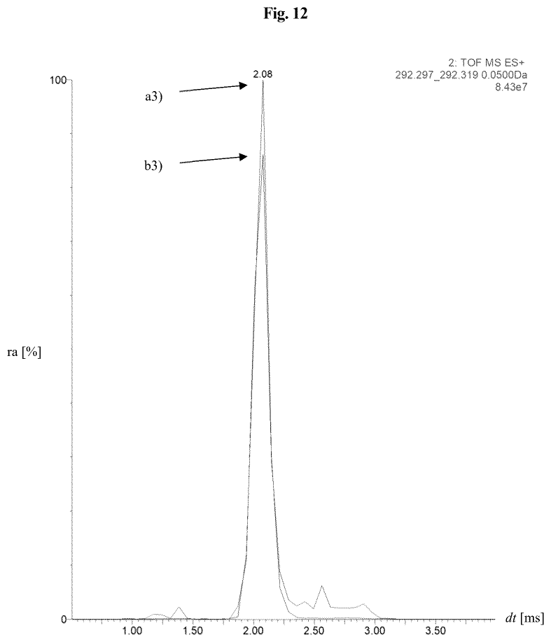

FIG. 12 shows the overlay of the extracted ion mobilograms (0.5 to 4.0 ms drift time dt) of Testosterone-13C3 [M+H]⁺ mass signal at m/z 292.3 detected after microparticle enrichment, extraction and ionization starting from neat solution a3) and additionally spiked in horse serum b3). Starting concentrations were both 10 ng/mL. To every analyte solution in wellplate (100 μL), 15 μL BeadA suspension (10 mg/mL) was added and incubated for 3 min. After magnetic separation and removal of the supernatant, the analyte-microparticle complexes were washed 2× with water (100 μL). The residual analyte-bead complexes were then extracted with 80% ACN+0.1% FA (10 μL) and subsequently ionized by nanoESI with a chip based instrument (Triversa NanoMate, Advion Inc.). The mass analysis of the resulting ions was performed with a Synapt G2-Si mass spectrometer (Waters Corp.) in combination with ion mobility separation (IMS; wave velocity 650 m/s, wave height 40 V) in the IMS-ToF positive ion mode with an acquisition time of 60 s. Despite the complex matrix, starting from analyte spiked horse serum resulted in similar counts of Testosterone-13C3 detected, compared to the same experiment from neat analyte solution. This highlights the superior capabilities of enriching and purifying analytes of interest out of complex matrices that would not be possible to measure via direct infusion out of initial samples. The comparison of the detection of Testosterone-13C3, applying the microparticle based sample enrichment/purification with extraction and ionization in the positive ion mode, considering a neat solution versus horse serum matrix is shown.

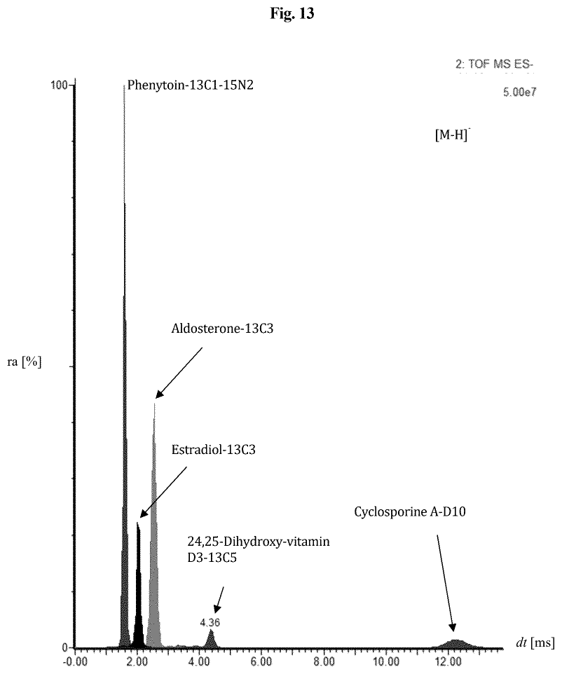

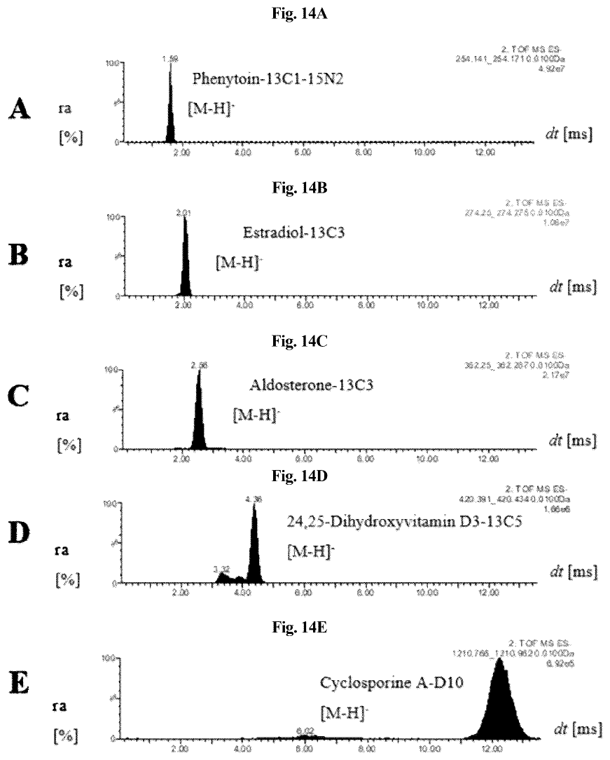

FIGS. 13 and 14A-E show the ion mobility separation of different analytes in a mixture, applying the microparticle based sample enrichment with extraction and ionization in the negative ion mode.

FIGS. 14A-14E show the overlay of five extracted ion mobilograms (0 to 14 ms drift time dt) detected after microparticle enrichment, extraction and ionization out of a single analyte mix. All five substances represent important diagnostic and therapeutic analytes, representing Phenytoin-13C1-15N2, Estradiol-13C3, Aldosterone-13C3, 24,25 Dihydroxyvitamin D3-13C5 and Cyclosporine A-D10 and are spiked to final concentrations of 10 ng/mL (100 μL, neat solution), except Aldosterone-13C3, that was contained in 20 ng/mL. To the analyte-mix solution in wellplate, 15 μL BeadA suspension (10 mg/mL) was added and incubated for 3 min. After magnetic separation and removal of the supernatant, the analyte-microparticle complex was washed 2× with water (100 μL). The residual analyte-microparticle complex was then extracted with 80% ACN+0.08 mM NH₄F+NH₄OH pH=9.0 (10 μL) and subsequently ionized by nanoESI with a chip based instrument (Triversa NanoMate, Advion Inc.). The separation and mass analysis of the resulting ions was performed with a Synapt G2-Si mass spectrometer (Waters Corp.) in combination with ion mobility separation (IMS; wave velocity 650 m/s, wave height 40 V) in the IMS-ToF positive ion mode with an acquisition time of 60 s. All spiked analytes were enriched and extracted successfully out of one single analyte mix. The analytes appeared in their corresponding proton loss [M−H]⁻.

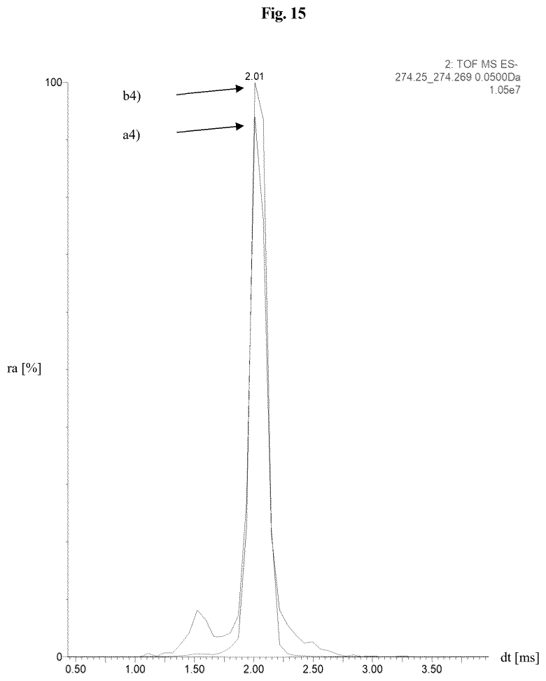

FIG. 15 shows the comparison of the detection of Estradiol-13C3, applying the microparticle based sample enrichment/purification with extraction and ionization in the negative ion mode, considering a neat solution versus horse serum matrix. FIG. 15 shows the overlay of the extracted ion mobilograms (0.5 to 4.0 ms drift time) of Estradiol-13C3 [M−H]⁻ mass signal at m/z 274.3 detected after microparticle enrichment, extraction and ionization starting from neat solution a4) and additionally spiked in horse serum b4). Starting concentrations were both 10 ng/mL. To every analyte solution in wellplate (100 μL), 15 μL BeadA suspension (10 mg/mL) was added and incubated for 3 min. After magnetic separation and removal of the supernatant, the analyte-microparticle complexes were washed 2× with water (100 μL). The residual analyte-microparticle complexes were then extracted with 80% ACN+0.08 mM NH₄F+NH₄OH pH=9.0 (10 L) and subsequently ionized by nanoESI with a chip based instrument (Triversa NanoMate, Advion Inc.). The mass analysis of the resulting ions was performed with a Synapt G2-Si mass spectrometer (Waters Corp.) in combination with ion mobility separation (IMS; wave velocity 650 m/s, wave height 40 V) in the IMS-ToF negative ion mode with an acquisition time of 60 s. Despite the complex matrix, starting from analyte spiked horse serum resulted in similar counts of Estradiol-13C3 detected, compared to the same experiment from neat analyte solution. This highlights the superior capabilities of enriching and purifying analytes of interest out of complex matrices that would not be possible to measure via direct infusion out of initial samples.

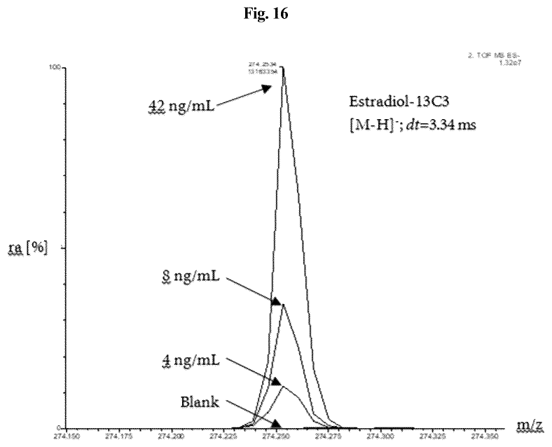

FIG. 16 shows the application of microparticle, e.g. magnetic immunobeads, for the detection of Estradiol-13C3, applying the microparticle sample enrichment with extraction and ionization in the negative ion mode. Estradiol-13C3 from neat analyte mix; microparticle workflow with anti-Estradiol antibody conjugated superparamagnetic immunobeads (“iBead(E2)”). Analyte concentrations are 42 ng/mL, 8 ng/mL, 4 ng/mL Estradiol-13C3 and compared to blank water. To every analyte solution in wellplate (100 μL), 10 μL iBead(E2) suspension (11 mg/mL) was added and incubated for 10 min. After magnetic separation and removal of the supernatant, the analyte-microparticle complex was washed 2× with water (100 μL). The residual analyte-microparticle complexes were then extracted with 80% ACN+0.08 mM NH₄F+NH₄OH pH=9.0 (10 L) and subsequently ionized by nanoESI with a chip based instrument (Triversa NanoMate, Advion Inc.). The mass analysis of the resulting ions was performed with a Synapt G2-Si mass spectrometer (Waters Corp.) in combination with ion mobility separation (IMS; wave velocity 950 m/s and wave height 40 V) in the IMS-ToF negative ion mode with an acquisition time of 60 s. FIG. 16 shows an overlay of the extracted full scan mass spectra of the relevant [M−H]⁻ signal of Estradiol-13C3 at a drift time of 3.34 ms. The extraction and ionization of Estradiol-13C3 directly from immunobeads was successfully for the tested concentrations of 42 ng/mL, 8 ng/mL and 4 ng/mL, while the blank spectrum showed almost no background signals in the observed mass region.

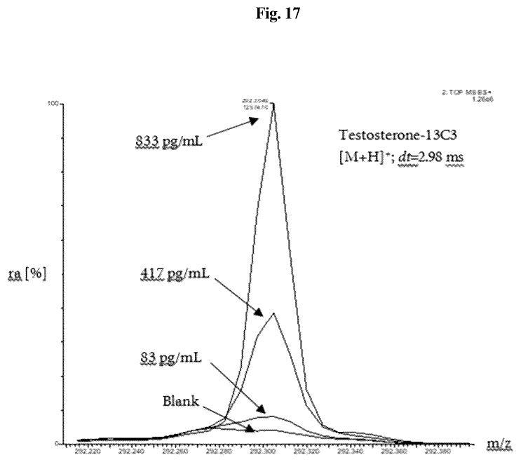

FIG. 17 shows an application of microparticle, e.g. magnetic immunobeads, for the detection of Testosterone-13C3, applying the microparticle based sample enrichment with extraction and ionization in the positive ion mode. Testosterone-13C3 from neat analyte mix; microparticle workflow with anti-Testosterone antibody conjugated superparamagnetic immunobeads (“iBead(Te)”). Analyte concentrations are 833 pg/mL, 417 pg/mL, 83 pg/mL Testosterone-13C3 and compared to blank water. To every analyte solution in wellplate (100 μL), 15 μL iBead(Te) suspension (4 mg/mL) was added and incubated for 10 min. After magnetic separation and removal of the supernatant, the analyte-microparticle complex was washed 2× with water (100 μL). The residual analyte-microparticle complexes were then extracted with 80% ACN+0.1% FA (10 μL) and subsequently ionized by nanoESI with a chip based instrument (Triversa NanoMate, Advion Inc.). The mass analysis of the resulting ions was performed with a Synapt G2-Si mass spectrometer (Waters Corp.) in combination with ion mobility separation (IMS; wave velocity 850 m/s and wave height 40 V) in the IMS-ToF positive ion mode with an acquisition time of 60 s. FIG. 17 shows an overlay of the extracted full scan mass spectra of the relevant [M+H]⁺ signal of Testosterone-13C3 at a drift time of 2.98 ms. The extraction and ionization of Testosterone-13C3 directly from microparticles, e.g. immunobeads, was successfully for the tested concentrations of 833 pg/mL and 417 pg/mL. Even at a concentration of 83 pg/mL, the signal is still higher compared to the blank HS iBead(Te) extract.

The method of the invention shows a valuable tool for measuring clinical important analytes in low concentrations. The successful example of an application using specific microparticle, e.g. immunobeads, highlights the modularity and broadness of applicability, as tailored microparticles, e.g. immunobeads, can aim the detection of analytes of interest that would not be possible otherwise. Additionally, different microparticle materials could easily be implemented on one instrument, targeting different analytes of interest, without changing the sample preparation as well as ionization process itself.

This patent application claims the priority of the European patent application 22211164.3, wherein the content of this European patent application is hereby incorporated by references.

### LIST OF REFERENCES

1—Analysis module
2—sample solution provided in assay cups
3—pipetting units
4—analyte solution with microparticle suspension
5—residual analyte adsorbed on the microparticle
6—analysis module
7—microparticle extraction module
8—microparticle separation module for bead workflow
9—microparticle extraction tips
10—microparticle extraction solvent reservoir
11—microparticle capturing plate
12—nanospray chip
13—analysis module inlet

1. A method of determining the presence or the level of an analyte of interest in a sample by a chip based nanoESI detection system, wherein the chip based nanoESI detection system comprises an electrically conductive pipette tip and a nano-electrospray nozzle, said method comprises the following steps:
a) Providing the sample including the analyte of interest and a matrix, wherein the matrix is non-magnetic,
b) Providing a microparticle, wherein the microparticle is magnetic,
c) Incubation of the microparticle and the analyte of interest to form an analyte-microparticle complex in a sample holder, wherein the analyte-microparticle complex is magnetic,
d) Separating the matrix and the analyte-microparticle complex by magnetic forces,
e) Optionally washing the analyte-microparticle complex in the sample holder,
f) Extracting the analyte from the analyte-microparticle complex by an extraction solvent and magnetic forces, said step (f) comprises
f1) Providing the extraction solvent by the electrically conductive pipette tip,
f2) Contacting the extraction solvent and the analyte-microparticle complex in the sample holder,
f3) Extracting the analyte of interest from the analyte-microparticle complex to form an extracted analyte of interest, wherein the microparticle is retained in the sample holder by magnetic forces during the extracting step 3), wherein the electrically conductive pipette tip comprises the extracted analyte of interest,
g) Directly contacting the electrically conductive pipette tip comprising the extracted analyte of interest and the nano-electrospray nozzle of a chip based nanoESI detection system to form a nano-electrospray for ionization of the extracted analyte of interest,
h) Determining the presence or the level of the extracted analyte of interest in the sample using the chip based nanoESI detection system, wherein the chip based nanoESI detection system uses mass spectrometry, ion mobility and/or a combination thereof.

2. The method of claim 1, wherein the electrically conductive pipette tip comprising the extracted analyte of interest is free of a microparticle.

3. The method of claim 1, wherein the material of the electrically conductive pipette tip comprises an electrically conductive material selected from the group consisting of at least partially graphene, carbon nanotubes, carbon black, carbon fibers, stainless steel, aluminum, titanium, chromium, electrically conductive metals and alloys thereof.

4. The method of claim 1, wherein the electrical conductive pipette tip comprises a microparticle content with respect to the total content of the microparticle which is less than 20%, 15%, 10%, 8%, 6%, 4%, 2%, 1%, 0.1% or 0.01%.

5. The method of claim 1, wherein the directly contact between the electrically conductive pipette tip and the nozzle of a chip based nanoESI detection system is a directly electrically contact.

6. The method of claim 1, wherein the microparticle is supramagnetic or paramagnetic.

7. The method of claim 1, wherein the matrix comprises analyte-interfering components derived from biological samples, microparticle, sample preparation solutions, mixtures or combinations thereof.

8. The method of claim 1, wherein the matrix is a solution.

9. The method of claim 1, wherein the method is automated and/or is performed in a random-excess mode.

10. The method of claim 1, wherein the method is free of a chromatographic step comprises at least one or more methods selected from the following group: chromatography, high performance liquid chromatography (HPLC), liquid chromatography high performance liquid chromatography (LC-HPLC), gas chromatography (GC), gel permeation chromatography (GPC), flash chromatography.

11. Use of the method of claim 1, for determining the presence or the level of an analyte of interest in a sample.

12. A diagnostic system for determining the presence or the level of an analyte of interest in a sample, comprising a chip based nanoESI source, an electrically conductive pipette tip and a detector to carry out the method according to claim 1, wherein the chip based nanoESI source comprises a nozzle, wherein the detector uses mass spectrometry or ion mobility or combination thereof.

13. Use of the diagnostic system of claim 12 in a method of determining the presence or the level of the analyte of interest in the sample by a chip based nanoESI detection system, wherein the chip based nanoESI detection system comprises an electrically conductive pipette tip and a nano-electrospray nozzle, said method comprises the following steps:
a) Providing the sample including the analyte of interest and a matrix, wherein the matrix is non-magnetic,
b) Providing a microparticle, wherein the microparticle is magnetic,
c) Incubation of the microparticle and the analyte of interest to form an analyte-microparticle complex in a sample holder, wherein the analyte-microparticle complex is magnetic,
d) Separating the matrix and the analyte-microparticle complex by magnetic forces,
e) Optionally washing the analyte-microparticle complex in the sample holder,
f) Extracting the analyte from the analyte-microparticle complex by an extraction solvent and magnetic forces, said step (f) comprises
f1) Providing the extraction solvent by the electrically conductive pipette tip,
f2) Contacting the extraction solvent and the analyte-microparticle complex in the sample holder,
f3) Extracting the analyte of interest from the analyte-microparticle complex to form an extracted analyte of interest, wherein the microparticle is retained in the sample holder by magnetic forces during the extracting step 3), wherein the electrically conductive pipette tip comprises the extracted analyte of interest,
g) Directly contacting the electrically conductive pipette tip comprising the extracted analyte of interest and the nano-electrospray nozzle of a chip based nanoESI detection system to form a nano-electrospray for ionization of the extracted analyte of interest,
h) Determining the presence or the level of the extracted analyte of interest in the sample using the chip based nanoESI detection system, wherein the chip based nanoESI detection system uses mass spectrometry, ion mobility and/or a combination thereof.

14. A kit suitable to perform a method of claim 1 comprising
(A) a microparticle for enriching or purification the analyte of interest in a sample,
(B) an extraction solvent for extracting the analyte of interest from the microparticle,
(C) optionally an internal standard, and
(D) optionally a catalyst or other reagents, e.g. derivatization reagents.

15. Use of a kit of the preceding claim 14 in a method of determining the presence or the level of the analyte of interest in the sample by a chip based nanoESI detection system, wherein the chip based nanoESI detection system comprises an electrically conductive pipette tip and a nano-electrospray nozzle, said method comprises the following steps:
a) Providing the sample including the analyte of interest and a matrix, wherein the matrix is non-magnetic,
b) Providing a microparticle, wherein the microparticle is magnetic,
c) Incubation of the microparticle and the analyte of interest to form an analyte-microparticle complex in a sample holder, wherein the analyte-microparticle complex is magnetic,
d) Separating the matrix and the analyte-microparticle complex by magnetic forces,
e) Optionally washing the analyte-microparticle complex in the sample holder,
f) Extracting the analyte from the analyte-microparticle complex by an extraction solvent and magnetic forces, said step (f) comprises
f1) Providing the extraction solvent by the electrically conductive pipette tip,
f2) Contacting the extraction solvent and the analyte-microparticle complex in the sample holder,
f3) Extracting the analyte of interest from the analyte-microparticle complex to form an extracted analyte of interest, wherein the microparticle is retained in the sample holder by magnetic forces during the extracting step 3), wherein the electrically conductive pipette tip comprises the extracted analyte of interest,
g) Directly contacting the electrically conductive pipette tip comprising the extracted analyte of interest and the nano-electrospray nozzle of a chip based nanoESI detection system to form a nano-electrospray for ionization of the extracted analyte of interest,
h) Determining the presence or the level of the extracted analyte of interest in the sample using the chip based nanoESI detection system, wherein the chip based nanoESI detection system uses mass spectrometry, ion mobility and/or a combination thereof.
